SEQUENCE LISTING (18 sequences)

SEQ ID NO: 1 - DNA, 2412 bp, unidentified
  source 1..2412: mol_type = unassigned DNA
atgaaaacagttgttataatagatgcacttagaacacctataggaaagtataaaggatcacttagccaggttagtgcagttgatttaggaacacatgttacaacacaattacttaaaagacatagtacaataagtgaagaaatagatcaagttatatttggaaatgttcttcaagctggaaatggacagaatcctgcaagacagatagcaataaatagcggacttagccatgaaatacctgcaatgacagttaatgaagtttgcggaagtggaatgaaagcagttatacttgcaaagcaacttatacagcttggagaagcagaagttttaatagctggaggaatagaaaatatgagtcaagcacctaaacttcaaagatttaattatgaaacagaaagttatgatgcaccttttagtagtatgatgtatgatggacttacagatgcatttagtggacaagcaatgggacttactgcagaaaatgttgcagagaaatatcatgttactagagaagaacaagatcaatttagtgttcatagtcaacttaaggcagcacaagcacaagctgaaggtatatttgctgatgaaatagcacctcttgaggttagtggaacacttgttgaaaaagatgagggaataagacctaatagttcagttgagaagcttggaactcttaaaacagtttttaaagaagatggaacagttacagcaggtaatgcatcaacaataaatgatggtgcatcagcacttataatagcaagccaagaatatgctgaggcacatggattaccttatcttgcaataataagagatagtgttgaagttggaatagatcctgcatatatgggaataagcccaataaaggcaatacaaaaacttcttgctagaaatcaacttacaactgaagaaattgatttatatgaaataaatgaagcatttgcagcaacaagtatagttgttcagagagaattagcacttcctgaagaaaaagttaatatatatggtggtggaataagtcttggacatgcaattggagcaacaggtgcaagattacttacaagccttagctatcaacttaatcaaaaagaaaagaaatatggtgttgcaagtctttgcattggaggtggattaggattagcaatgttacttgaaagacctcaacaaaagaaaaatagtagattttatcaaatgagtccagaagaaagattagcatcacttcttaatgaaggacaaataagtgcagatacaaagaaagaatttgaaaatacagcacttagtagtcaaatagcaaatcatatgatagaaaatcaaatatcagaaactgaagttcctatgggagttggacttcatttaacagttgatgaaactgattatcttgttccaatggcaacagaggaacctagtgttatagcagcattaagtaatggtgcaaagatagcacagggatttaaaactgttaatcaacagagacttatgagaggacagatagtattttatgatgttgctgatcctgaaagtcttatagataaattacaagttagagaagctgaagtatttcaacaggcagagcttagttatcctagtatagttaaaagaggtggaggacttagagatcttcaatatagaacatttgatgaatcatttgtttctgtagattttcttgttgatgttaaagatgcaatgggagctaatatagttaatgcaatgcttgaaggtgttgcagaactttttagagaatggtttgcagaacaaaagatactttttagtatacttagtaattatgcaactgaatcagtagttacaatgaaaactgcaatacctgttagtagactttcaaaaggatcaaatggaagagaaatagcagaaaagatagttcttgcaagtagatatgcatcacttgatccttatagagcagttacacataataagggaataatgaatggaatagaagcagttgttcttgctactggaaatgatacaagagcagtttcagcaagttgtcatgcatttgctgttaaagagggaagatatcagggacttacaagttggacattagatggtgaacagcttattggagagataagtgttcctcttgcattagcaacagttggaggtgcaacaaaagttcttcctaaaagtcaagcagcagcagatttacttgcagttactgatgcaaaagaacttagtagagttgttgcagcagttggattagcacaaaatcttgcagctttaagagcacttgttagcgagggaattcaaaaaggacatatggcattacaggcaagatcacttgcaatgactgttggtgcaactggaaaagaagttgaagcagtagcacaacaacttaagagacaaaagacaatgaatcaagatagagcaatggcaatacttaatgatcttagaaaacaataa

SEQ ID NO: 2 - DNA, 1152 bp, unidentified
  source 1..1152: mol_type = unassigned DNA
atgacaataggaatagataagatttcattttttgttcctccatattatattgatatgacagcacttgctgaagcaagaaatgttgatcctggaaaatttcatataggaattggacaggatcaaatggcagttaatcctataagccaagatatagttacatttgcagctaatgcagcagaagcaatacttacaaaagaggataaagaagctatagatatggttatagttggaacagaaagtagtatagatgaaagcaaagcagcagcagttgttcttcatagacttatgggaatacagccttttgcaagaagttttgaaataaaagaagcatgttatggtgcaacagcaggacttcaacttgcaaagaatcatgttgcacttcatcctgataaaaaggttcttgttgttgcagcagatatagcaaaatatggacttaatagtggtggagagcctacacaaggtgctggtgcagttgcaatgttagttgcaagtgaacctagaatacttgcacttaaagaagataatgttatgcttacacaagatatatatgatttttggagacctacaggacatccttatcctatggttgatggacctcttagcaatgaaacatatatacaaagttttgcacaagtttgggatgaacataaaaagagaacaggacttgattttgcagattatgatgcacttgcttttcatataccttatacaaaaatgggaaagaaagcacttttagcaaaaataagtgatcaaacagaggcagaacaagaaagaatattagcaagatatgaagaaagtatagtttatagtagaagagttggaaatctttatacaggatctctttatcttggattaatatcacttcttgaaaatgcaacaactcttacagctggaaatcaaataggactttttagttatggaagcggagcagttgcagagttttttacaggtgaattagttgcaggatatcaaaatcatcttcaaaaagaaacacatcttgcattacttgataatagaacagaactttcaatagcagaatatgaagcaatgtttgcagaaacacttgatacagatatagatcaaacattagaagatgaacttaagtatagtataagtgcaataaataatacagttagaagttatagaaattaa

SEQ ID NO: 3 - DNA, 906 bp, unidentified
  source 1..906: mol_type = unassigned DNA
atggttagttgtagtgcacctggaaagatttatctttttggagagcatgcagttgtttatggtgaaacagcaatagcatgtgcagttgagttaagaacaagagttagagcagaacttaatgatagtataacaatacaaagtcaaataggaagaacaggacttgattttgaaaaacatccttatgttagtgcagttatagaaaagatgagaaaaagtatacctataaatggtgtttttcttacagttgatagtgatatacctgttggaagtggattaggaagtagtgcagcagttacaatagcaagtattggagcacttaatgaactttttggatttggactttcacttcaagaaatagcaaagcttggacatgaaatagaaataaaagttcaaggtgcagcaagtcctacagatacatatgttagtacatttggaggtgttgttacaatacctgaaagaagaaagcttaaaacacctgattgtggaatagttattggagatacaggtgtatttagtagtacaaaagaacttgttgcaaatgttagacaacttagagaaagttatcctgatcttatagaacctcttatgacaagtataggaaaaataagtagaattggagaacaacttgtacttagcggagattatgcaagcataggtagacttatgaatgttaatcaaggacttcttgatgcacttggagttaatatacttgaacttagtcaacttatatatagtgcaagagcagctggtgcatttggagcaaaaataacaggtgcaggtggtggtggatgtatggttgcattaacagcacctgaaaagtgtaatcaagttgcagaggcagttgctggtgcaggtggaaaagttacaataacaaaacctacagagcaaggacttaaagttgattaa

SEQ ID NO: 4 - DNA, 1188 bp, unidentified
  source 1..1188: mol_type = unassigned DNA
atgacagtttatacagcaagcgttacagcacctgttaatatagcaacacttaagtattggggaaaaagagatacaaaacttaatcttcctacaaatagtagtataagtgttacactttcacaagatgatcttagaacacttacaagtgcagcaacagcaccagaatttgagagagatacactttggttaaatggtgaacctcatagtatagataatgaaagaacacaaaattgtcttagagatcttagacagcttagaaaagaaatggaatcaaaagatgcatcacttcctacacttagtcagtggaagcttcatatagttagtgaaaataattttcctacagcagcaggacttgcatcaagtgctgcaggatttgcagcattagttagtgcaatagcaaaactttatcaacttcctcaaagtacaagtgaaataagtagaatagcaagaaaaggatcaggatcagcatgtagaagtctttttggaggatatgttgcatgggaaatgggaaaagctgaagatggacatgatagtatggcagttcaaatagcagatagtagtgattggcctcaaatgaaggcatgtgttcttgttgttagtgatataaagaaagatgttagtagtacacagggaatgcagcttacagttgcaacaagcgaactttttaaagaaagaatagaacatgttgttcctaaaagatttgaagttatgagaaaagcaatagttgaaaaggattttgcaacatttgctaaagaaacaatgatggatagtaatagttttcatgcaacttgtcttgatagttttcctcctattttttatatgaatgatacaagtaaaagaataataagttggtgtcatacaataaatcaattttatggtgaaacaatagttgcatatacatttgatgcaggacctaatgcagtactttattatcttgcagaaaatgaaagtaaactttttgcatttatttataagctttttggaagtgttcctggatgggataagaagtttacaacagaacagcttgaagcatttaatcatcaatttgaaagtagtaattttacagcaagagaacttgatcttgaacttcaaaaagatgttgcaagagttatacttacacaagttggaagtggacctcaagagacaaatgagagtcttatagatgcaaagacaggacttcctaaagaa

SEQ ID NO: 5 - DNA, 1353 bp, unidentified
  source 1..1353: mol_type = unassigned DNA
atgagcgagcttagagcatttagtgcacctggaaaagcattattagcaggtggatatcttgttcttgatacaaaatatgaagcatttgttgttggacttagtgcaagaatgcatgcagttgcacatccttatggaagtttacaaggatcagataaatttgaagttagagttaaaagtaaacaatttaaagatggtgaatggctttatcatataagtcctaaaagtggatttatacctgttagtattggaggaagtaaaaatccttttatagaaaaagttatagcaaatgtttttagctattttaagcctaatatggatgattattgtaatagaaatctttttgttatagatatatttagtgatgatgcatatcattcacaagaagattcagttacagaacatagaggaaatagaagattaagttttcatagtcatagaatagaagaagttcctaaaacaggacttggaagtagtgcaggattagttacagttcttacaacagcacttgcatcattttttgttagtgatcttgaaaataatgttgataagtatagagaagttatacataatcttgcacaagttgctcattgtcaagcacagggaaaaataggatcaggatttgatgttgcagcagcagcatatggaagtataagatatagaagatttcctcctgcacttatatcaaatcttccagatataggatctgcaacatatggatcaaagcttgcacatcttgttgatgaagaagattggaatataacaataaaaagtaatcatcttcctagtggacttacactttggatgggagatataaagaatggaagcgagacagttaagcttgttcaaaaagttaagaattggtatgatagtcatatgcctgaaagtcttaagatatatacagaacttgatcatgcaaatagtagatttatggatggactttcaaaacttgatagacttcatgaaacacatgatgattatagtgatcagatatttgaatcacttgaaagaaatgattgtacatgtcaaaagtatcctgaaataactgaagtaagagatgcagtagcaacaataagaagatcatttagaaaaataacaaaagaaagcggagctgatatagagcctcctgttcaaacaagtcttttagatgattgccagacacttaaaggtgttcttacatgtttaatacctggtgcaggtggttatgatgcaatagcagtaataacaaagcaagatgttgatcttagagcacaaacagcaaatgataaaagatttagtaaagttcaatggcttgatgttacacaggcagattggggagttagaaaagaaaaagatccagaaacatatcttgataaa

SEQ ID NO: 6 - DNA, 1860 bp, unidentified
  source 1..1860: mol_type = unassigned DNA
atgagctttgatatagcaaaatatcctacacttgcacttgttgatagcacacaagagcttagattacttcctaaagagagccttcctaagctttgtgatgagcttagaagatatttacttgatagtgttagtagaagtagtggacattttgcaagtggacttggaacagttgaacttacagttgcacttcattatgtttataatacaccttttgatcaacttatatgggatgttggacatcaagcatatcctcataagatacttactggaagaagagataaaataggaacaataagacaaaaaggtggacttcatccttttccatggagaggtgaaagtgagtatgatgttttaagcgttggacatagtagtacaagcataagtgctggaataggaatagcagttgcagcagaaaaagagggtaagaatagaagaacagtttgtgttattggagatggtgcaataacagctggaatggcatttgaagcaatgaatcatgctggtgatataagacctgatatgttagttatacttaatgataatgaaatgagtataagtgaaaatgttggagcacttaataatcatcttgcacaattacttagtggaaaactttatagtagtcttagagaaggtggaaagaaagtttttagtggtgttcctcctataaaagaacttcttaaaagaacagaggaacatataaaaggtatggttgttcctggaacactttttgaagaacttggatttaattatataggacctgttgatggacatgatgttcttggacttataacaacacttaagaatatgagagatcttaaaggacctcaatttcttcatataatgacaaagaaaggtagaggatatgagcctgctgaaaaggatcctataacatttcatgcagttccaaaatttgatcctagcagtggatgtttacctaagagtagtggtggattacctagttatagtaagatatttggagattggctttgcgagacagcagcaaaagataataaacttatggcaataacaccagcaatgagagaaggatctggaatggttgaatttagtagaaaatttcctgatagatattttgatgttgcaatagcagagcaacatgcagttacatttgcagcaggattagcaattggaggatataagccaatagttgcaatatatagtacatttcttcaaagagcatatgatcaagtacttcatgatgttgctatacaaaaacttccagttctttttgcaatagatagagcaggtatagttggagcagatggacaaacacatcaaggtgcatttgatcttagctatttaagatgtatacctgaaatggttataatgactcctagtgatgagaatgagtgcagacagatgctttatacaggatatcattataatgatggacctagtgcagttagatatcctagaggaaatgcagttggagtagaacttacacctcttgagaaacttccaataggaaagggaatagttaagagaagaggtgagaagcttgcaatacttaattttggtacacttatgcctgaggcagctaaagttgcagaaagtttaaatgcaacacttgtagatatgagatttgttaaacctcttgatgaagctcttatattagaaatggcagcaagccatgaagcacttgttacagttgaagagaatgcaataatgggaggtgcaggatcaggtgtaaatgaagttttaatggcacatagaaaacctgtacctgttcttaatataggacttcctgatttttttatacctcagggaacacaagaagaaatgagagcagaattaggattagatgcagcaggtatggaagcaaagattaaagcatggttagca

SEQ ID NO: 7 - DNA, 1194 bp, unidentified
  source 1..1194: mol_type = unassigned DNA
atgaagcagcttacaatacttggaagtacaggatcaataggatgtagtacacttgatgttgttagacataatcctgaacattttagagttgttgcacttgttgctggaaagaatgttacaagaatggttgaacagtgtcttgaattttcacctagatatgcagttatggatgatgaggcaagtgcaaagcttcttaagacaatgcttcaacaacagggaagcagaacagaagttttatcaggacaacaagcagcatgtgatatggcagcattagaagatgttgatcaagttatggcagctatagttggagcagcaggattattacctacacttgcagcaataagagctggaaaaacaatacttcttgcaaataaagaaagtcttgttacatgtggtagactttttatggatgcagttaagcaaagtaaagcacaacttcttccagttgatagcgaacataatgcaatatttcagagccttcctcagcctatacaacataatttaggatatgcagatcttgaacaaaatggtgttgttagtatacttcttactggaagcggaggaccttttagagaaacacctttaagagatcttgcaactatgacacctgatcaagcatgtagacatcctaattggagtatgggaagaaaaataagtgttgattcagcaacaatgatgaataaaggacttgaatatatagaagcaagatggctttttaatgcaagtgcatcacagatggaagttcttatacatcctcaaagtgttatacatagtatggttagatatcaagatggatcagttcttgcacaacttggagaacctgatatgagaacacctatagcacatacaatggcatggcctaatagagttaatagcggagttaagcctcttgatttttgcaagcttagcgcattaacatttgcagcacctgattatgatagatatccttgccttaagcttgctatggaagcatttgaacaaggacaagcagctacaacagcacttaatgcagcaaatgaaataacagttgcagcttttcttgctcaacaaataagatttacagatatagcagctcttaatcttagtgttcttgaaaagatggatatgagagaacctcagtgcgttgatgatgttttaagcgttgatgcaaatgcaagagaagttgctagaaaagaagttatgagattagcaagt

SEQ ID NO: 8 - DNA, 531 bp, unidentified
  source 1..531: mol_type = unassigned DNA
atggcaataactggaatattttttggaagtgatactggaaatacagaaaatatagcaaaaatgatacaaaaacaacttggaaaagatgttgcagatgttcatgatatagctaaaagtagtaaagaagatcttgaagcatatgatatacttttattaggaatacctacatggtattatggtgaggcacaatgtgattgggatgatttttttcctacacttgaagaaatagattttaatggaaaacttgttgcactttttggatgcggagatcaagaggattatgcagagtatttttgtgatgcacttggaacaataagagatataatagagcctagaggtgcaacaatagttggacattggcctacagcaggatatcattttgaagcaagtaaaggacttgcagatgatgatcattttgttggacttgctatagatgaggatagacagcctgaattaacagcagaaagagttgaaaagtgggttaaacaaataagtgaagaacttcatcttgatgaaatacttaatgcataa

SEQ ID NO: 9 - DNA, 1119 bp, unidentified
  source 1..1119: mol_type = unassigned DNA
atgcataatcaggcacctatacaaagaagaaaaagtacaagaatatatgttggaaatgttcctattggagatggtgcacctatagcagttcaaagcatgacaaatacaagaacaactgatgttgaagcaacagttaatcaaataaaagcacttgaaagagttggagcagatatagttagagttagcgttcctacaatggatgcagcagaagcatttaagcttataaaacaacaagttaatgttcctcttgttgcagatatacattttgattatagaatagcacttaaagttgcagaatatggtgttgattgtcttagaataaatcctggaaatataggaaatgaagaaagaataagaatggttgtagattgtgcaagagataagaatattcctataagaattggagttaatgcaggatcacttgaaaaagatcttcaagaaaagtatggtgagcctacacctcaagcattattagaaagtgcaatgagacatgttgatcatcttgatagacttaattttgatcaatttaaagttagtgttaaagcatcagatgtttttcttgcagttgaaagttatagacttcttgcaaaacaaatagatcaacctcttcatcttggaataactgaagcaggtggtgcaagatcaggtgcagttaaatcagcaataggacttggattacttcttagtgaaggtataggtgatacacttagagtttcattagcagcagatcctgttgaagagataaaagttggatttgatatacttaaatcacttagaattagaagtagaggaataaattttatagcatgtccaacatgtagtagacaagaatttgatgttataggaacagtaaatgctcttgaacaaagacttgaagatataataacacctatggatgtttcaataataggatgtgttgttaatggacctggtgaggcattagttagtacacttggagttacaggtggaaataagaaaagcggactttatgaggatggtgttagaaaagatagacttgataataatgatatgatagatcaattagaagcaagaattagagcaaaagcaagccagttagatgaagctagaagaatagatgttcagcaagttgaaaaataa

SEQ ID NO: 10 - DNA, 1209 bp, unidentified
  source 1..1209: mol_type = unassigned DNA
atgcagacattacctagtcctgttcaagcaacacctacagaaacagcaatagttagaagaaaaacaagacctgttccaataggatcagttgttattggaggtggacatcctgttgcagttcagagtatgataaatgaagatacacttgatatagaaggatcagtagcagcaattagaagattacatgaaataggatgtgaaatagtaagagttacagttcctagtcttgcacatgcaaaagcaatggaagagataagagatagactttataagacatataaacctgttcctcttgttgcagatgttcatcataatggaatgaaaatagctcttgaagttgcaaaatatgttgataatgttagaataaatcctggactttatgtttttgaaaaaccaaaacctaatagaacagaatatacacaagcagaatttgatgaaattggagcaaaaataagagaaacacttgaacctcttgttataagtcttagagatcagggaaaaagtatgagaattggagttaatcatggaagtcttgcagaaagaatgctttttacatatggtgatacacctgaaggtatggttgaatcagcacttgagtttataagaatttgtgaatcacttaatttttataatcttgaaatatcacttaaagcaagtagagttcctgttatgatagcagctaatagacttatggttaaaagaatggatgaacttggaatggattatcctcttcatcttggagttactgaagctggtgatggtgaatatggaagaataaaaagcacagctggaatagcaacacttttagcagaaggtattggagatacaataagagttagccttacagaggcacctgagaaagaaatacctgtttgctatggaatacttcaagcacttggacttagaagaacaatggttgagtatgttgcatgtcctagttgtggaagaacactttttaatcttgaggaagttcttcataaagttagagaagcaacaaaacatttaacaggacttaatattgcagttatgggatgtatagttaatggacctggtgaaatggcagatgcagattatggatatgttggaaagcagcctggatatataagcctttatagaggaagagaagaagttaaaaaggttcctgaagctgaaggtgttgcagcattagttgaacttataaaggcagatggaagatgggttgatccttaa

SEQ ID NO: 11 - DNA, 1209 bp, unidentified
  source 1..1209: mol_type = unassigned DNA
atggatacaaagacatttaagagaacacttcaacatagtgaaaattataatagaaaaggatttggacatcaagcagaagttgcaacacaacttcaaagcgaatatcaaagtagtcttatacaagaaataagagatagaaattatacacttcaaagaggagatgttacaataagacttgcacaagcatttggattttgctggggagttgaaagagcagttgcaatggcatatgaaacaagaaaacattttcctacagaaagaatatggataacaaatgaaataatacataatcctagtgttaatcaaagaatgcaagaaatgcaagttggatttatacctgttgaagctggaaataaagattttagtgttgttggaaataatgatgttgttatacttcctgcatttggagcaagcgttcaagagatgcaacttttaagtgaaaaaggatgtaaaatagttgatacaacatgtccttgggttagtaaagtttggaatacagttgaaaaacataaaaagggtgatcatacaagtataatacatggaaagtataagcatgaagaaacaatagcaacaagtagttttgctggaaaatatcttatagttcttaatcttaaagaagcacaatatgttgcagattatatacttcatggtggaaatagagaagaatttcttcaaaaatttgcaaaagcatgtagtgcaggatttgatcctgatagagatcttgagagagttggaatagcaaatcaaacaacaatgcttaagggtgaaacagaacaaataggaaaactttttgaacatacaatgttacaaaaatatggaccagttgaacttaatcaacattttcaaagttttaatacaatatgtgatgcaactcaagaaagacaagatgcaatgcttgaacttgttcaagaaaatcttgatcttatgatagttattggaggatttaatagtagtaatacaacacaattacaacaaatatcacaagaaagaggacttccaagttatcatatagatgttgttgaaagaataaaatcaataaatagtatagaacatagacaacttaatggtgaacttgtaacaacagaaaattggcttcctgctggaaagatagtagttggagttacaagtggtgcaagtacacctgataaagttgttgaggatgttatagaaaagatatttgcacttaaagcaacagcagcagtattttaa

SEQ ID NO: 12 - DNA, 294 bp, unidentified
  source 1..294: mol_type = unassigned DNA
atggcaacatataaggttacacttgttagacctgatggaagtgaaacaacaatagatgttcctgaagatgaatatatacttgatgttgcagaggaacaaggacttgatcttccttttagttgtagagctggtgcatgtagtacatgtgctggaaaattacttgaaggtgaggttgatcaaagtgatcaatcatttcttgatgatgatcaaatagaaaaaggatttgttcttacatgtgttgcatatcctagaagtgattgtaaaatacttacaaatcaagaagaagaattatat

SEQ ID NO: 13 - DNA, 1158 bp, unidentified
  source 1..1158: mol_type = unassigned DNA
atgtataatgcaacaaatagtagaagtagaatgtttagatatgaagttgttggacttagacaaacagcagaaacagaaaagacaaattatgcaataagaaatagtggatctcaattttttaatgttccttatgatagaatgaatcaatttatgcagcaaataacaagatggggaggaaaaatagttagtatacaacctcttaatggaacagttgcacctttagcagcaacaacagaacctgcagctaataatggtgcagcacctgttaaagaaaagaaagttgatatacctgttaatatatatagacctaataatccttgtattggaaaagttataagtaatgaagaacttgttagagaaggtggtgagggaacagttaaacatataatatttgatataagtggaacagaacttagatatcttgaaggacaaagtataggaataatacctgctggaactgatgcaaatggaaagcctcataagcttagactttatagtatagcaagtacaagacatggtgattttcaagatgataaaacagttagtctttgtgttagaagattagaatataaggataaagaaactggagaaactatatatggtgtttgtagtagttatcttaatcaacttcagcctggagatgaagtaaagataacaggacctgttggaaaagaaatgcttcttagtgatgatcctgaagcaacaataataatgcttgcaactggaactggaatagcaccttttagagcttttctttggagaatgtttaaagaaaataatcctgattatcaatttaaaggacttgcatggttattttttggagttgcatatacagcaaatatactttataaggatgaacttgaagcaatacaagcacaatatcctgatcattttagacttacatatgcaatatcaagagaacaaaagacacctgatggtggaaaaatgtatattcagggaagaatagcagagcatgcagatgaaatatggcaacttcttcaaaagaaaaatacacatgtttatatgtgtggattaagaggaatggaacctggaattgatgaagcaatgacagctgcagcagcaaagaatggtgctgattggcaagaatttcttaagggaacacttaagaaagaaggtagatggcatgttgaaacatat

SEQ ID NO: 14 - DNA, 864 bp, unidentified
  source 1..864: mol_type = unassigned DNA
atgacagcagataataatagtatgcctcatggtgcagtttcaagttatgcaaagcttgttcaaaatcaaacacctgaagatatacttgaagaatttcctgaaataatacctcttcaacaaagacctaatacaagaagtagtgaaacaagtaatgatgaaagcggagaaacttgttttagcggacatgatgaggaacaaataaaacttatgaatgaaaattgtatagttcttgattgggatgataatgcaattggagctggaacaaagaaagtttgtcatcttatggaaaatatagaaaaaggacttcttcatagagcatttagtgtttttatatttaatgaacaaggtgaacttcttttacaacaaagagcaacagaaaagataacatttcctgatctttggacaaatacatgttgtagccatccactttgcatagatgatgaattaggacttaagggaaaacttgatgataagataaaaggtgcaataacagcagcagttagaaaattagatcatgaacttggaataccagaagatgaaactaaaactagaggaaaatttcattttcttaatagaatacattatatggcacctagtaatgagccttggggagaacatgagatagattatatattattttataagattaatgcaaaagaaaatcttacagttaatcctaatgttaatgaagttagagattttaagtgggttagtcctaatgatcttaaaacaatgtttgcagatcctagttataagtttacaccttggtttaagattatatgtgaaaattatctttttaattggtgggaacagcttgatgatcttagcgaagttgaaaatgatagacagatacatagaatgtta

SEQ ID NO: 15 - DNA, 1635 bp, unidentified
  source 1..1635: mol_type = unassigned DNA
atggaagctagaagatcagcaaattatgaacctaatagttgggattatgattatcttcttagtagtgatacagatgaaagtatagaagtttataaagataaagcaaagaaacttgaagcagaagttagaagagaaataaataatgaaaaagcagaatttcttacacttcttgaacttatagataatgttcaaagacttggacttggatatagatttgaaagtgatataagaggtgcacttgatagatttgttagtagcggaggatttgatgcagttacaaagacaagtttacatggaacagcacttagttttagattacttagacaacatggatttgaagttagtcaagaagcattttcaggatttaaagatcaaaatggaaattttcttgaaaatcttaaagaagatataaaagcaatacttagtctttatgaagcaagttttcttgcacttgaaggtgaaaatatacttgatgaagcaaaagtttttgcaataagtcatttaaaagaactttcagaagaaaagataggaaaagaattagcagaacaagttaatcatgcattagaacttccacttcatagaagaacacagagattagaagcagtttggagcatagaggcatatagaaagaaagaggatgcaaatcaggttttattagaattagctatattagattataatatgatacaaagtgtttatcaaagagatcttagagaaacaagtagatggtggagaagagttggattagcaacaaagcttcattttgcaagagatagacttatagaaagtttttattgggcagttggagttgcatttgagcctcaatatagtgattgtagaaatagtgttgcaaaaatgttttgttttgttacaataatagatgatatatatgatgtttatggaactcttgatgaacttgaactttttacagatgcagttgaaagatgggatgttaatgcaataaatgatcttcctgattatatgaaactttgctttcttgctctttataatactataaatgaaatagcatatgataatcttaaggataagggtgagaatattcttccttatcttacaaaggcatgggcagatctttgcaatgcatttcttcaagaggcaaaatggttatataataaaagtacacctacatttgatgattattttggaaatgcatggaaaagtagtagcggtcctcttcaacttgtttttgcatattttgcagttgttcagaatataaagaaagaagaaatagaaaatcttcaaaaatatcatgatacaataagtagacctagtcatatatttagactttgtaatgatcttgcaagtgcatcagcagaaatagcaagaggtgaaacagcaaatagcgttagctgttatatgagaacaaagggaataagcgaggaacttgcaacagagtcagttatgaatcttattgatgaaacatggaaaaagatgaataaagaaaaacttagtggaagtctttttgctaaaccttttgttgaaacagctataaatcttgctagacaaagtcattgtacatatcataatggtgatgcacatacaagccctgatgagcttacaagaaaaagagttctttcagttataacagaacctatacttccttttgaaagataa

SEQ ID NO: 16 - DNA, 1464 bp, unidentified
  source 1..1464: mol_type = unassigned DNA
ttacttcatttttgtattaaaactatataagaaaaaatatacatagtatatatactttataaaacaactttataaaggcttttgtggggatatagtatatggtacaatatgaaggtaaagtaaaatactagtgattaaggttggtgcgatatgtttgatatagaccagaattccaataagggtaaaattttaaacttgttatatagaaaaagagagcttactaaacttgatatatcaaatgagattggtgtaagtgttccaacagtaatatccaatgtgaatgaacttataagagaaggctttgtagaagaatcaggagtagcaagctctacagggggaaggaaacctataatagtgagctttattccaaatagtagatatgcttttggtgtagatataaatccatatatggcaagagtagttttaactaatcttgacttagaaattaagtatgatgttgagtttttaatagaagagcttaaggatattaaatctataatgaaaaggatatcgcttatagttaaagaggctctggagtttacaaagattgatgagaagaaggtacttggaattggattctcacttcaaggaacagttgatgaggaaaatctagtactcaaaagagctatcaatataggagttaaggactgggattttaaattgtttgaaaacctatttcaatttcctctatttattgaaaacgaagcaaacgctgcagcacttgcagagctaaaaataggtatagcaaaagaggagagaaatcttgtctatatatcagtttcaagcggattaggtactggaatagtgattgagggacagctttataagggaaagaacaagagagctggagaaatgggtcatatgaccttagttcctcatggaaaactgtgcagatgtgggagaaaagggtgctgggaaatgtatgcatctcaaaaagctcttttagatgaatttaatgaattaagtgatgtaaaggttactaagttagagcagttttttaatctaataatgataaaaaataaagttgcagagaaatgtctaaataattacttaaacaatcttgcatcaggtatacaaaatatagttctaatttttgaccctcattatatagttctagggggagaaataagtggatattctgatgtttatttagatagtcttaaggataaaattttcgatgaaaatgagttttataatagaggtgattttaagctattcccatctaggcttaaaaggaattcctcaattttaggagcggctttattgcctatacaaaaagcatttttaattgaacaagatgtaatttgaaattagtaaacacaggtaaatataaatagtattgtattttaatagatgatagaaaaagcataggagacttattgaggaaagtcttcttaaaatcatttatgtaaacgatttaatttaactagtgaaatagggggtaaaaaataatgggatggccgg

SEQ ID NO: 17 - DNA, 191 bp, unidentified
  source 1..191: mol_type = unassigned DNA
ggccgctttatgaattttatatacataaatgagctttaaatactatcatcacaattctaaggatacctgatttattcttttttatagacttaggaaattttatagttcaaactttttaaagtgctttttaaaagttattgaaaaagtttgtatgcaatgatataatttaacatgtaaataattacaggtac

SEQ ID NO: 18 - DNA, 599 bp, unidentified
  source 1..599: mol_type = unassigned DNA
gcagccgaacgaccgagcgcagcgagtcagtgagcgaggaagcggaagagcgcctgatgcggtattttctccttacgcatctgtgcggtatttcacaccgcatatgatgagtgagacctgagctcagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctataatgtgaattcgcggccgcttctagacatgggttattccttatctatctaactagtagcggccgctgcaggcatgcgagagtagggaactgccaggcatcaaataaaacgaaaggctcagtcgaaagactgggcctttcgttttatctgttgtttgtcggtgaacgctctcctgagtaggacaaatccgccgggagcggat

(19) United States
(12) Patent Application Publication
(10) Pub. No.: US 2026/0117135 A1
(43) Pub. Date: Apr. 30, 2026

(54) SYSTEMS AND METHODS FOR PRODUCING A DIMETHYLCYCLOOCTANE-BASED AVIATION FUEL FROM ISOPRENE

(71) Applicant: CleanJoule, Inc., Salt Lake City, UT (US)

(72) Inventors: Robert Andrew Price, Salt Lake City, UT (US); Mukund Karanjikar, Salt Lake City, UT (US); Oyvind Hatlevik, West Valley City, UT (US); Michael James Mella, South Jordan, UT (US); Gregory Clarence Turpin, Salt Lake City, UT (US); Younghwan Kim, Salt Lake City, UT (US); Jonathan Cloyd Greenhalgh, Salt Lake City, UT (US); Tsokyi Choera, Salt Lake City, UT (US); Christopher Paul Smith, Bozeman, MT (US)

(73) Assignee: CleanJoule, Inc., Salt Lake City, UT (US)

(21) Appl. No.: 18/928,691

(22) Filed: Oct. 28, 2024

(51) Int. Cl. C10L 1/08; C07C 2/46; C07C 7/00; C12N 9/02; C12N 9/10; C12N 15/70

(52) U.S. Cl. CPC C10L 1/08; C07C 2/46; C07C 7/005; C12N 9/0008; C12N 9/1085; C12N 15/70; C12Y 102/04001; C12Y 205/0101; C07C 2523/34; C07C 2523/42; C07C 2523/44; C07C 2523/656; C07C 2523/745; C07C 2523/75; C07C 2523/755; C07C 2525/02; C10L 2270/04; C12N 2800/101

(57) ABSTRACT

System and method of producing a dimethylcyclooctane-based aviation fuel from isoprene includes culturing a genetically engineered microbe under suitable conditions to produce a first product including isoprene, dimerizing, using a transition metal-based catalyst, isoprene to form a second product including dimethylcyclooctadiene, and hydrogenating, using a hydrogenation catalyst, dimethylcyclooctadiene to form a third product including dimethylcyclooctane, wherein the genetically engineered microbe includes a heterologous nucleic acid construct encoding a peptide, the peptide having an activity configured to contribute to an enhanced production of isoprene.

### STATEMENT OF GOVERNMENT SUPPORT

This invention was made with government support under DE-SC0013230 and DE-EE0008504, awarded by the United States Department of Energy. The government has certain rights in the invention.

### FIELD OF THE INVENTION

The present invention generally relates to the field of metabolic engineering. In particular, the present invention is directed to system and method for producing a dimethylcyclooctane-based aviation fuel from isoprene.

### REFERENCE TO SEQUENCE LISTING

This specification includes a sequence listing submitted herewith, which includes the file entitled 1656-001USUI_Sequence_Listing.xml having the following size: 36,456 bytes which was created Sep. 4, 2024, the contents of which are incorporated by reference herein.

### BACKGROUND

Aviation fuels such as Jet A-1 and aviation gasoline typically contain hydrocarbon molecules with 4-16 carbon atoms. These fuels are often produced from unsustainable fossil fuels and/or using resource-intensive methods, such as by refining/cracking crude oil. Aviation fuels from biological sources, while having lower carbon footprint, often require extensive land use and energy input, which limits their overall sustainability. Sustainable and scalable means of generating aviation fuels remain elusive.

### SUMMARY OF THE DISCLOSURE

In an aspect, a method of producing a dimethylcyclooctane-based aviation fuel from isoprene is described. Method includes culturing a genetically engineered microbe under suitable conditions to produce a first product including isoprene, wherein the genetically engineered microbe includes a heterologous nucleic acid construct encoding a peptide, the peptide having an activity configured to contribute to an enhanced production of isoprene. Method further includes dimerizing, using a transition metal-based catalyst, isoprene to form a second product including dimethylcyclooctadiene. Method further includes hydrogenating, using a hydrogenation catalyst, dimethylcyclooctadiene to form a third product including dimethylcyclooctane.

These and other aspects and features of nonlimiting embodiments of the present invention will become apparent to those skilled in the art upon review of the following description of specific nonlimiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a schematic illustration of an exemplary embodiment of a method for producing a dimethylcyclooctane-based aviation fuel from isoprene; and

FIG. 2 is an exemplary scheme of the methylerythritol phosphate (MEP) pathway, the mevalonate (MVA) pathway, and additional metabolic pathways associated thereto that are relevant for biosynthesis of isoprene;

FIGS. 3A-B are additional exemplary schemes of expanded metabolic pathways that are relevant for biosynthesis of isoprene; potential gene targets are also included, wherein the gene targets may be overexpressed or knocked down/knocked out to improve the yield of isoprene;

FIG. 3C is an exemplary embodiment of experimental results collected using genetically engineered Escherichia Coli (E. Coli) with downregulated ackA-pta, poxB, and IdhA genes shown in FIGS. 3A-B;

FIG. 4 is an exemplary embodiment of an absorbing and stripping apparatus that may be used for producing a dimethylcyclooctane-based aviation fuel from isoprene;

FIG. 5A is an exemplary embodiment of general genetic sequences for the MEP and MVA pathways;

FIG. 5B is an exemplary embodiment of MVA1, MVA2, MEP1, and MEP2 regulator-promoter sequences of gene clusters, wherein the gene clusters encode specific enzymes along the MVA and MEP pathways;

FIG. 5C is an exemplary embodiment of various plasmid vector designs for MVA1, MVA2, MEP1, and MEP2 sequences of gene clusters in FIG. 5B;

FIG. 5D is an exemplary embodiment of a detailed vector map of a pCJ24-1 plasmid vector; and FIG. 5E is an exemplary embodiment of pCJ24-1 plasmid vectors shown in FIG. 5D that contains MVA1, MVA2, MEP1, and MEP2 gene clusters;

FIGS. 6A-B are exemplary embodiments of experimental results including isoprene titers measured by gas chromatography (GC) 24 h and 48 h after induction of IspA knock-down strains; the plot contains three groups (A-C) with four sub-groups organized by arabinose induction concentrations (0-1.8 wt %); groups A and B target the same area on an IspA-encoding gene; data are collected from triplicated cultures; the average values and error bars are calculated based on three independent measurements from such triplicated cultures; raw data are also displayed as black dots on each bar; statistical analyses are conducted using an independent sample t-test (*p≤0.05, **p<0.01); the t-test is one-tailed and assumes equal variances;

FIGS. 6C-D are exemplary embodiments of experimental results including isoprene titers measured by gas chromatography (GC) 24 h and 48 h after induction of AceE knock-down strains, consistent with details of FIGS. 6A-B;

FIG. 6E is an exemplary embodiment of experimental results including isoprene titers measured based on IspA knock-down strains, isoprene titers measured based on AceE knock-down strains, and isoprene titers measured based on a traditional fermentation protocol using a CJ23 reference strain (n=54); the mean values of isoprene titers for the CJ23 reference strain are 1,608 and 2,791 pA*s for the 24-hour and 48-hour fermentation periods, respectively; these values are statistically analyzed by t-test using mean values of IspA knock-down strains and AceE knock-down strains; for IspA knock-down strains, the average GC areas are 13345 and 12528 pA*s for the 24-hour and 48-hour fermentation periods, respectively; for AceE knock-down strains, the average GC areas are 8,535 and 11,013 pA*s for the 24-hour and 48-hour fermentation periods, respectively; statistical analyses are conducted using an independent sample t-test (*p≤0.05, **p≤0.01); the t-test is two-tailed and assumes unequal variances;

FIG. 6F is an exemplary embodiment of a conceptual image of asRNA location within the genomic DNA of a target gene; the first part (Part 1) targets a ribosome binding site (RBS) and an upstream region of the ATG, the second part (Part 2) centers on the ATG, and the last part (Part 3) includes the ATG and a downstream region;

FIGS. 6G-J are exemplary embodiments of plasmid vector maps for the CJ23 strain (pCJ24-2, pCJ24-3, pCJ24-4) and a modified pCJ24-4 vector for genetic knockdown;

FIG. 6K is an exemplary embodiment of a sequence composition of a target knockdown sequence within a promoter and/or a terminator;

FIG. 7A is an exemplary embodiment of experimental results showing isoprene production levels, indicated by integrated gas chromatography (GC) peak areas, in genetically engineered E. Coli strains with and without enzymes along the MVA pathway; each point represents a unique biological sample (colony); and

FIG. 7B is an exemplary embodiment of experimental results from two independent trials showing integrated GC peak areas measured after every hour of fermentation over the course of 80 hours.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods of producing a dimethylcyclooctane-based aviation fuel from isoprene.

Method includes culturing a genetically engineered microbe under suitable conditions to produce a first product including isoprene, wherein the genetically engineered microbe includes a heterologous nucleic acid construct encoding a peptide, the peptide having an activity configured to contribute to an enhanced production of isoprene. In one or more embodiments, genetically engineered microbe may include bacteria or fungi. In one or more embodiments, genetically engineered microbe may include Escherichia Coli, Vibrio natriegens, Saccharomyces cerevisiae, Aspergillus, Yarrowia lipolytica, Bacillus subtilis, Clostridium ljungdahlii, and/or Pseudomonas putida.

In one or more embodiments, method further includes purifying first product, using a first purification apparatus, to isolate isoprene. In some cases, first purification apparatus may include an absorbing and stripping apparatus. In some cases, absorbing and stripping apparatus may contain activated carbon.

Method further includes dimerizing isoprene, using a transition metal-based catalyst, to form a second product including dimethylcyclooctadiene. In one or more embodiments, transition metal-based catalyst may include an iron (Fe)-based catalyst, a cobalt (Co)-based catalyst, a nickel (Ni)-based catalyst, or a manganese (Mn)-based catalyst. In one or more embodiments, transition metal-based catalyst may include an organometallic catalyst. In one or more embodiments, transition metal-based catalyst may include a triphenylphosphine (TPP)-based ligand, a trialkylphosphine ligand, a trialkylphosphite ligand, an organophosphate ligand, and/or a malononitrile ligand, among others. In one or more embodiments, dimethylcyclooctadiene may include 1,5-dimethyl-1,5-cyclooctadiene, 1,6-dimethyl-1,5-cyclooctadiene, 2,6-dimethyl-1,3-cyclooctadiene, 2,6-dimethyl-1,4-cyclooctadiene, 3,7-dimethyl-1,5-cyclooctadiene, 3,7-dimethyl-1,3-cyclooctadiene, and/or 3,6-dimethyl-1,3-cyclooctadiene, among others.

In one or more embodiments, method may further include purifying second product, using a second purification apparatus, to isolate dimethylcyclooctadiene from at least a byproduct. In some cases, at least a byproduct may include at least a Diels-Alder [4+2] cycloaddition product. In some cases, second purification apparatus may include a distillation apparatus. In some cases, second purification apparatus may include a filtration apparatus.

Method further includes hydrogenating dimethylcyclooctadiene, using a hydrogenation catalyst, to form a third product including dimethylcyclooctane. In one or more embodiments, hydrogenation catalyst may include without limitation platinum(IV) oxide (PtO₂), Pd supported on carbon (Pd/C), rhenium—nickel (Re—Ni) alloy, nickel—aluminum (Ni—Al) alloy, Raney nickel, Ni supported on silica-alumina (SiO₂—Al₂O₃), among others. In one or more embodiments, dimethylcyclooctane may include cis-1,4-dimethylcyclooctane, trans-1,4-dimethylcyclooctane, cis-1,5-dimethylcyclooctane, and/or trans-1,5-dimethylcyclooctane, among others.

In one or more embodiments, method may further include filtrating third product, using a first filter, to form a first purified product. In some cases, first filter may include a pore size between 100 nanometers and 10 micrometers. In some cases, first filter may include a pore size of approximately 1 micrometer.

In one or more embodiments, method may further include filtrating first purified product, using a second filter, to form a second purified product. In some cases, second filter may include a clay filter.

Aspects of the present disclosure may be used to provide a sustainable means of producing aviation sources. Aspects of the present disclosure may be used to reduce the dependence on fossil fuels and mitigate the negative environmental impacts thereof. Exemplary embodiments illustrating aspects of the present disclosure are described below in the context of several specific examples.

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

It is understood that the acts described below are meant as a general overview and demonstration of an exemplary method, and that the method may include different and/or additional acts as described herein or otherwise.

While the present invention will be described as having particular configurations disclosed herein, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

It is to be understood that any aspect and/or element of any embodiment of the method(s) described herein or otherwise may be combined in any way to form additional embodiments of the method(s) all of which are within the scope of the method(s).

Where a process is described herein, those of ordinary skill in the art will appreciate that the process may operate without any user intervention. In another embodiment, the process includes some human intervention (for example, a step is performed by or with the assistance of a human).

For the purposes of this disclosure, including the claims, the phrase “at least some” means “one or more” and includes the case of only one. Thus, for example, the phrase “at least some ABCs” means “one or more ABCs” and includes the case of only one ABC.

For the purposes of this disclosure, including the claims, the term “at least one” should be understood as meaning “one or more” and therefore includes both embodiments that include one or multiple components. Furthermore, dependent claims that refer to independent claims that describe features with “at least one” have the same meaning, both when the feature is referred to as “the” and “the at least one”.

For the purposes of this disclosure, the term “portion” means some or all. Therefore, for example, “A portion of X” may include some of “X” or all of “X”. In the context of a conversation, the term “portion” means some or all of the conversation.

For the purposes of this disclosure, including the claims, the phrase “using” means “using at least” and is not exclusive. Thus, for example, the phrase “using X” means “using at least X”. Unless specifically stated by use of the word “only”, the phrase “using X” does not mean “using only X”.

For the purposes of this disclosure, including the claims, the phrase “based on” means “based in part on” or “based, at least in part, on” and is not exclusive. Thus, for example, the phrase “based on factor X” means “based in part on factor X” or “based, at least in part, on factor X”. Unless specifically stated by use of the word “only”, the phrase “based on X” does not mean “based only on X”.

In general, for the purposes of this disclosure, including the claims, unless the word “only” is specifically used in a phrase, it should not be read into that phrase.

For the purposes of this disclosure, including the claims, the phrase “distinct” means “at least partially distinct”. Unless specifically stated, distinct does not mean fully distinct. Thus, for example, the phrase “X is distinct from Y” means that “X is at least partially distinct from Y” and does not mean that “X is fully distinct from Y”. Thus, for the purposes of this disclosure, including the claims, the phrase “X is distinct from Y” means that X differs from Y in at least some way.

It should be appreciated that the words “first”, “second”, and so on, in the description and claims, are used to distinguish or identify, and not to show a serial or numerical limitation.

Similarly, letter labels (for example, “(A)”, “(B)”, “(C)”, and so on, or “(a)”, “(b)”, and so on) and/or numbers (for example, “(i)”, “(ii)”, and so on) are used to assist in readability and to help distinguish or identify, and are not intended to be otherwise limiting or to impose or imply any serial or numerical limitations or orderings. Similarly, words such as “particular”, “specific”, “certain”, and “given”, in the description and claims, if used, are to distinguish or identify, and are not intended to be otherwise limiting.

For the purposes of this disclosure, including the claims, the terms “multiple” and “plurality” mean “two or more,” and include the case of “two”. Thus, for example, the phrase “multiple ABCs” means “two or more ABCs” and includes “two ABCs”. Similarly, for example, the phrase “multiple PQRs” means “two or more PQRs” and includes “two PQRs”.

The present invention also covers the exact terms, features, values, and ranges, etc., in case these terms, features, values, and ranges, etc., are used in conjunction with terms such as “about”, “around”, “generally”, “substantially”, “essentially”, “at least”, etc. Thus, for example, “about 3” or “approximately 3” shall also cover exactly 3, and “substantially constant” shall also cover exactly constant.

For the purposes of this disclosure, unless stated otherwise, the terms “about” or “approximately” refer to a value that is within 10% above or below the value being described.

For the purposes of this disclosure, including the claims, singular forms of terms are to be construed as also including the plural form and vice versa, unless the context indicates otherwise. Thus, it should be noted that for the purposes of this disclosure, the singular forms “a”, “an”, and “the” include plural references unless the context clearly dictates otherwise. In other words, terms such as “a”, “an”, and “the” are not intended to refer to only a singular entity but include the general class of which a specific example may be used for illustration.

Throughout the description and claims, the terms “comprise”, “including”, “having”, “contain”, and their variations should be understood as meaning “including but not limited to” and are not intended to exclude other components unless specifically so stated.

For the purposes of this disclosure, the terms “administration” or “administering” refer to a method of giving a dosage of a compound or pharmaceutical composition to a subject.

For the purposes of this disclosure, the terms “treat”, “treating”, or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (for example, by reducing one or more symptoms of a neurological disorder). The term “therapeutic” includes the effect of mitigating deleterious clinical effects of certain processes (i.e., consequences of the process, rather than the symptoms of processes).

It will be appreciated that variations to the embodiments of the invention can be made while still falling within the scope of the invention. Alternative features serving the same, equivalent, or similar purpose can replace features disclosed in the specification, unless stated otherwise. Thus, unless stated otherwise, each feature disclosed represents one example of a generic series of equivalent or similar features.

Use of exemplary language, such as “for instance”, “such as”, “for example” (“for example,”), and the like, is merely intended to better illustrate the invention and does not indicate a limitation on the scope of the invention unless specifically so claimed.

While the invention has been described in connection with what is presently considered to be the most practical and embodiments and is further described in the examples below, it is to be understood that the invention is not to be limited to the disclosed embodiment, but on the contrary, is intended to cover various modifications and equivalent arrangements included within the spirit and scope of the appended claims.

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, and/or components have not been described in detail in order to avoid unnecessarily obscuring claimed subject matter. The illustrative embodiments described in the detailed description and claims are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and make part of this disclosure.

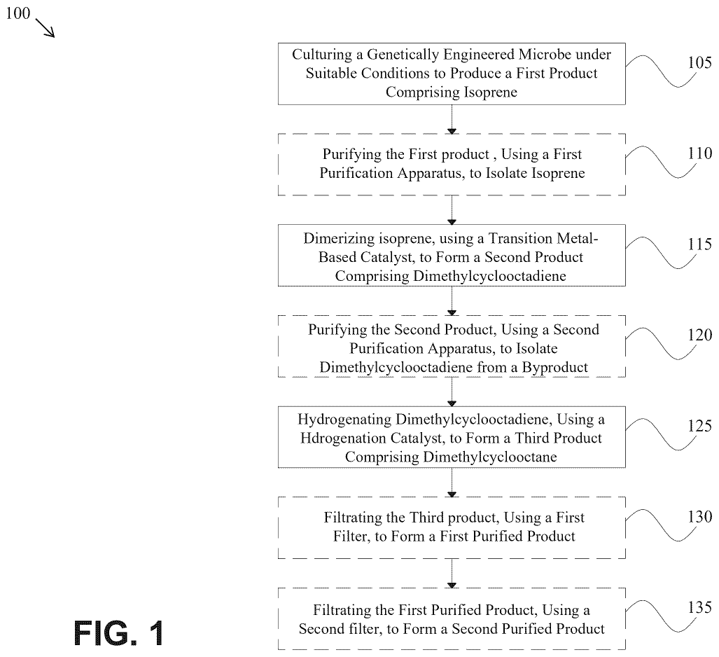

Referring now to FIG. 1, an exemplary embodiment of method 100 for producing a dimethylcyclooctane-based aviation fuel from isoprene is illustrated. At step 105, method 100 includes culturing a genetically engineered microbe under suitable conditions. For the purposes of this disclosure, a “genetically engineered microbe” is a microbe whose genetic material has been manipulated to alter one or more of its hereditary traits. Such manipulation may include without limitation inserting, deleting, or otherwise modifying one or more specific DNA sequences; as a result of such manipulation, a genetically modified microbe may exhibit one or more different traits in its structure, function, or the like, compared to its naturally occurring (i.e., wild-type) counterparts. For the purposes of this disclosure, a “microbe” is a microscopic organism, including bacteria, archaea, fungi, protozoa, viruses, and/or the like, that is capable of being utilized in one or more aspects of a biotechnological application. A microbe may be characterized by its ability to perform certain biological processes, such as without limitation fermentation, gene expression, and metabolite production, among others. A microbe may be harnessed for purposes such as without limitation recombinant protein production, bioremediation, synthesis of pharmaceuticals, and development of biofuels, among others, due to their diverse metabolic capabilities and ease of genetic manipulation. For the purposes of this disclosure, a “suitable condition” is an environmental condition or factor suitable for the growth and/or replication of a microbe. In some cases, a suitable condition may vary from one type of microbe to another. A suitable condition may include without limitation a suitable temperature/temperature range, a suitable pressure/pressure range, a suitable pH or pH range, a suitable ionic strength/range of ionic strength, a suitable osmolarity/range of osmolarity, a suitable osmotic pressure/range of osmotic pressure, a suitable concentration/concentration range of one or more nutrients, and/or a suitable level of metabolic waste/metabolites, among others. A person of ordinary skill in the art, upon reviewing the entirety of this disclosure, will be able to identify suitable conditions specific to one or more microbes described herein.

With continued reference to FIG. 1, in one or more embodiments, genetically engineered microbe may include bacteria or fungi. In one or more embodiments, genetically engineered microbe may include Escherichia Coli (or E. coli), Vibrio natriegens (or V. natriegens), Saccharomyces cerevisiae (or S. cerevisiae), Aspergillus, Yarrowia lipolytica (Y. lipolytica), Bacillus subtilis (B. subtilis), Clostridium ljungdahlii ((. ljungdahlii), and/or Pseudomonas putida (P. putida), among others.

With continued reference to FIG. 1, for the purposes of this disclosure, Escherichia coli (E. coli) is a widely studied bacterium that serves as a crucial model organism in biotechnology and molecular biology. E. coli is a gram-negative, rod-shaped bacterium naturally found in the intestines of humans and other warm-blooded animals. The simplicity, well-characterized genetics, and rapid growth of E. coli, among other traits, make it a suitable host for genetic engineering applications. It is commonly used for producing recombinant proteins, plasmid cloning, and various metabolic engineering processes.

With continued reference to FIG. 1, for the purposes of this disclosure, Vibrio natriegens (V. natriegens) is a fast-growing, gram-negative marine bacterium. V. natriegens has recently gained prominence in biotechnology due to its exceptionally rapid doubling time of less than 10 minutes under suitable conditions. The fast growth, genetic tractability, high transformation efficiency, and ability to thrive in simple media of V. natriegens, among other traits, make it a suitable organism for various applications, including synthetic biology, recombinant protein production, metabolic engineering, and high-throughput screening, among others.

With continued reference to FIG. 1, for the purposes of this disclosure, Saccharomyces cerevisiae (S. cerevisiae) is a unicellular eukaryote, commonly known as brewer's yeast or baker's yeast, that plays a pivotal role in biotechnology due to its well-characterized genetics and ease of manipulation. S. cerevisiae has been instrumental in winemaking, baking, and brewing since ancient times and is a model system extensively used for recombinant DNA technology, metabolic engineering, and synthetic biology applications. Its ability to perform post-translational modifications similar to higher eukaryotes makes it suitable for producing complex proteins and biopharmaceuticals. Additionally, S. cerevisiae may be employed in biofuel production, fermentation processes, and various industrial biotechnology applications.

With continued reference to FIG. 1, for the purposes of this disclosure, Aspergillus is a genus of filamentous fungi. Aspergillus is characterized by its ability to produce a wide array of enzymes, organic acids, and secondary metabolites, making it a suitable host for production of biofuels, pharmaceuticals, and food products. Several genetic manipulation techniques have been established for Aspergillus. Certain Aspergillus species, such as Aspergillus niger and Aspergillus oryzae, may be used for recombinant protein production and metabolic engineering due to their efficient protein secretion systems and robust metabolic capabilities.

With continued reference to FIG. 1, for the purposes of this disclosure, Yarrowia lipolytica (Y. lipolytica) is a species of yeast belonging to genus Yarrowia and the family Dipodascaceae. It is known for its ability to utilize a wide range of carbon sources, particularly lipids, which makes it a useful organism in biotechnological applications. Yarrowia lipolytica may be used in the production of various bioproducts, including single-cell oils, enzymes, and organic acids. Yarrowia lipolytica is also notable for its potential use in bioremediation, as it may degrade environmental pollutants such as hydrocarbons and fatty acids. Yarrowia lipolytica has a well-studied genetic system, allowing for genetic modifications and metabolic engineering to enhance its production capabilities. Additionally, Yarrowia lipolytica is recognized for its lack of toxicity in food and industrial applications, making it an attractive candidate for use in various biotechnological processes.

With continued reference to FIG. 1, for the purposes of this disclosure, Bacillus subtilis (B. subtilis), is a Gram-positive, rod-shaped bacterium commonly found in soil and the gastrointestinal tracts of ruminants and humans. Bacillus subtilis is known for its ability to form endospores, which allow it to survive in harsh environmental conditions. Bacillus subtilis is widely studied for its role in various applications, including biotechnology, agriculture, and food production. Bacillus subtilis serves as a model organism for laboratory studies due to its genetic tractability and well-characterized physiology. Additionally, Bacillus subtilis may be utilized as a probiotic in animal feed and/or as a biocontrol agent in agriculture, promoting plant growth and/or protecting crops from pathogens. Its capability of producing enzymes, antibiotics, and/or other bioactive compounds further underscores its significance in various industrial processes.

With continued reference to FIG. 1, for the purposes of this disclosure, Clostridium ljungdahlii (C. ljungdahlii) is a gram-positive, anaerobic, rod-shaped bacterium that is part of the genus Clostridium. It is notable for its ability to perform autotrophic growth by utilizing a mixture of carbon monoxide (CO), carbon dioxide (CO₂), and hydrogen (H₂) (i.e., syngas) as sole carbon and energy sources, through a process known as the Wood-Ljungdahl pathway (acetyl-CoA pathway). Clostridium ljungdahlii is capable of converting syngas into value-added products, such as without limitation acetone, butanol, ethanol (ABE), and acetic acid, making it an important organism for industrial biotechnology applications, particularly in gas fermentation (for example, syngas fermentation) and biofuel production. Clostridium ljungdahlii holds promise for producing renewable fuels and chemicals from waste gases, such as those generated by industrial processes or gasified biomass. Specifically, an advantage of Clostridia as a host organism may derive from their ability to employ diverse carbon sources, including mono-, oligo- and polysaccharides that would be found in waste products, making the fermentation of industrial, agricultural and waste products conceivable.

With continued reference to FIG. 1, for the purposes of this disclosure, Pseudomonas putida (P. putida) is a gram-negative, rod-shaped, aerobic bacterium belonging to the genus Pseudomonas. Pseudomonas putida is commonly found in soil, water, and various environments where organic compounds are present. Pseudomonas putida is notable for its metabolic versatility, which allows it to degrade a wide range of organic compounds including without limitation hydrocarbons, aromatic compounds, and/or environmental pollutants such as without limitation toluene and naphthalene, making it an important organism for environmental bioremediation. Additionally, Pseudomonas putida may be used in various industrial and biotechnological applications due to its ability to produce valuable compounds and its resilience under harsh conditions. Pseudomonas putida may also be used to produce chemicals, such as biofuels, bioplastics, and other value-added compounds, through microbial fermentation and/or genetic engineering.

With continued reference to FIG. 1, in some cases, creating a genetically engineered microbe may involve using recombinant DNA technology, CRISPR-Cas9, gene cloning, and/or other molecular biology techniques to achieve desired phenotypic changes. For the purposes of this disclosure, “recombinant DNA technology” is a type of technology that involves a manipulation of DNA sequences to create new genetic combinations not found in nature. Recombinant DNA technology may include techniques such as gene cloning, insertion of DNA fragments from one organism into another, and/or use of vectors such as plasmids to transfer a genetic material. Recombinant DNA technology may be used to express new traits or produce biological products such as proteins, enzymes, and hormones and may play a pivotal role in fields such as genetic engineering, biotechnology, and pharmaceutical development.

With continued reference to FIG. 1, for the purposes of this disclosure, a “plasmid” is a circular, double-stranded DNA molecule distinct from a cell's chromosomal DNA and capable of autonomous replication. A plasmid may be used as a vector for insertion, expression, and propagation of foreign genes within a host organism. Such vectors may include specific sequences for an origin of replication, selectable markers, and cloning sites, enabling manipulation and study of genetic material for applications in research, biotechnology, and therapeutic development.

With continued reference to FIG. 1, for the purposes of this disclosure, “CRISPR-Cas9” is a genetic engineering tool, derived from a bacterial immune defense mechanism, that includes Clustered Regularly Interspaced Short Palindromic Repeats (CRISPR) sequences and the CRISPR-associated protein 9 (Cas9) enzyme and allows for precise, targeted modification of DNA within an organism. CRISPR-Cas9 may be programmed with a guide RNA to target a specific DNA sequence, enabling Cas9 enzyme to create double-strand breaks at one or more precise locations, thereby facilitating insertion, deletion, or modification of genetic material.

With continued reference to FIG. 1, genetically engineered microbe includes a heterologous nucleic acid construct. For the purposes of this disclosure, a “heterologous” nucleic acid construct is a nucleic acid construct that originates from a different species or is artificially synthesized and introduced into a host organism. A heterologous nucleic acid construct may be inserted into a host genome or expressed in a host cell to produce proteins, confer new traits, and/or study gene functions. A heterologous nucleic acid may include genes, regulatory elements, or other sequences that are not naturally found in the host organism's genome. For the purposes of this disclosure, a “nucleic acid construct” is a nucleic acid sequence that encodes a peptide and is capable of being expressed to synthesize the peptide. In other words, a nucleic acid construct includes a coding sequence that encodes a peptide and one or more regulatory elements that regulate the expression of the coding sequence. Exemplary embodiments of a regulatory element may include a promoter, an enhancer, a silencer, an insulator, an operator, and a response element, among others. For the purposes of this disclosure, a “promoter” is a DNA sequence where an RNA polymerase binds to initiate transcription. For the purposes of this disclosure, an “enhancer” is a distant element of DNA sequence that increases transcription rates by interacting with a promoter via DNA looping. For the purposes of this disclosure, a “silencer” is a DNA sequence that represses transcription when bound by specific proteins. For the purposes of this disclosure, an “insulator” is a DNA sequence that prevents the interaction between one or more enhancers and one or more promoters of neighboring genes. For the purposes of this disclosure, an “operator” is a DNA segment that regulates the transcription of adjacent genes. For the purposes of this disclosure, a response element is a DNA sequence that responds to external signals, allowing genes to be turned on or off in response to environmental changes. These regulatory elements may work together to ensure precise gene expression necessary for proper cellular function.

With continued reference to FIG. 1, in one or more embodiments, nucleic acid construct may include one or more operons. For the purposes of this disclosure, an “operon” is a functioning unit of DNA containing a cluster of genes under the control of a single promoter. It is commonly found in prokaryotes such as bacteria. These genes are transcribed together into a single messenger RNA strand and typically encode proteins that work together in a specific biological pathway. An operon may include a regulatory element, such as an operator as described above, where an activator or repressor protein may bind to increase or inhibit transcription. An operon may include one or more regulatory genes that encode one or more such activator or repressor proteins. A nonlimiting example of operon may include the lac operon, which regulates lactose metabolism in E. coli.

With continued reference to FIG. 1, nucleic acid construct encodes a peptide, the peptide having an activity configured to contribute to an enhanced production of isoprene. Additional details regarding possible mechanisms of such enhancement will be described below in this disclosure. In one or more embodiments, the activity of peptide may include a catalytic activity. For the purposes of this disclosure, a “catalytic activity” is the ability of a chemical to accelerate a chemical reaction by functioning as a catalyst. Specifically, a catalyst performs its catalytic function by lowering at least an activation barrier along a reaction coordinate and increasing at least a rate constant associated with the at least an activation barrier. In some cases, to perform a catalytic function, a catalyst may first be consumed by one or more reactants to form one or more intermediates, then be regenerated as the one or more intermediates are converted to one or more products. In some cases, one or more reactants may bind to a catalyst, participate in a chemical reaction, then dissociate from the catalyst as one or more products.

With continued reference to FIG. 1, in one or more embodiments, peptide may include an enzyme. For the purposes of this disclosure, an “enzyme” is a biological catalyst, often a protein, having a three-dimensional structure specifically tailored for fitting a substrate or reactant and catalyzing a chemical reaction therefrom. A substrate may interact with an enzyme (and its active/binding site) via a lock-and-key mechanism or an induced fit. Enzymes are often characterized by their high catalytic activity, high specificity toward substrates, and sensitivity to environmental factors such as temperature and pH. The catalytic activity of a catalyst or enzyme may be described using mathematical tools such as Arrhenius equation, Eyring equation. Michaelis-Menten equation, Lineweaver-Burk equation, among others, as deemed suitable by a person of ordinary skill in the art upon reviewing the entirety of this disclosure.

With continued reference to FIG. 1, genetically engineered microbe is configured to produce a first product. In some cases, first product may be referred to as “off-gas”. For the purposes of this disclosure, an “off-gas” a gas-phase substance, likely a mixture, that is released or vented from a chemical process, reaction, or industrial system. Off-gases often contain volatile compounds, unreacted gases, or reaction byproducts that are either unwanted or need to be processed, treated, or recovered. The composition of an off-gas may vary depending on the specific process, and it may include gases such as carbon dioxide (CO₂), carbon monoxide (CO), methane (CH₄), hydrogen (H₂), sulfur dioxide (SO₂), nitrogen (N₂), and volatile organic compounds (VOCs).

With continued reference to FIG. 1, first product includes isoprene. With continued reference to FIG. 1, for the purposes of this disclosure, “isoprene”, also known as 2-methyl-1,3-butadiene, is a volatile organic compound with the chemical formula C₅H₈, a structural formula of CH₂—CHC(CH₃)═CH₂, and a normal boiling point of 34° C. Isoprene may be considered a “hemiterpene” (i.e., half of a terpene), and the details of terpenes will be described below in this disclosure. Isoprene is a key building block for synthesizing natural rubber and various other polymers. Additionally, isoprene may be used in the manufacture of synthetic rubber, adhesives, and elastomers, as well as in the production of pharmaceuticals and other specialty chemicals. Isoprene may be produced naturally by plants and trees, particularly in large amounts by the rubber tree (i.e., Hevea brasiliensis). It may also be produced industrially through the thermal cracking of petroleum-derived naphtha or, in some cases, through biotechnological methods using genetically engineered microbes, as described in this disclosure. In some cases, isoprene may be synthesized using industrial organic chemistry, via synthetic routes such as an acetone/acetylene route, a propylene dimer route, an isoamylene route, an isopentane route, or an isobutylene/formaldehyde route, among others.

With continued reference to FIG. 1, for the purposes of this disclosure, a “terpene” is an organic compound with the general molecular formula (C₅H₈)ₙ, often characterized by a structure that contains repeating isoprene units. Terpenes are produced predominantly by plants, particularly conifers, and certain insects, and may be involved in various biological functions, including defense mechanisms and communication. Terpenes may be classified as monoterpenes (n=2), sesquiterpenes (n=3), diterpenes (n=4), and higher terpenes (n>4), based on the number of isoprene units they contain. Monoterpenes (C₁₀H₁₆) may include without limitation limonene found in citrus peels, myrcene in hops and lemongrass, pinene in pine resin, linalool in lavender, and geraniol in rose oil, among others. Sesquiterpenes (C₁₅H₂₄) may include without limitation farnesene in apple coatings, humulene in hops, bisabolol in chamomile, caryophyllene in black pepper, and nerolidol in ginger and tea tree oil. Diterpenes (C₂₀H₃₂) may include without limitation taxadiene from yew trees, gibberellins as plant hormones, phytol in chlorophyll, steviol in Stevia leaves, and retinol (Vitamin A1) derived from β-carotene. Higher terpenes may include without limitation squalene (C₃₀H₅₀) in shark liver oil, lanosterol (C₃₀H₅₀₀) in wool grease, tetraterpenes or tetraterpenoids including carotenoids (C₄₀H₅₆) such as beta-carotene in carrots, and rubber (polyterpene) from latex. Terpenes may serve as a biochemical precursor for a wide array of natural products, including essential oils, resins, and various pharmacologically active compounds, making them valuable in industries such as pharmaceuticals, perfumery, and agriculture. Isoprene belongs to a broader class of molecules called isoprenoids, and similarly, terpenes belong to a broader class of molecules called terpenoids, though it is worth noting that these terminologies may often be used interchangeably. An isoprenoid may include a molecule that is derived from isoprene, and similarly, a “terpenoid” may include a molecule that is derived from a terpene, consistent with details described above. Isoprenoids and terpenoids include a large and diverse class of naturally occurring organic compounds. Isoprenoids/terpenoids may also be synthesized through either the MEP pathway or the MVA pathway, as described in detail above. Isoprenoids/terpenoids may play essential roles in various biological processes, including without limitation cellular membrane structure (as cholesterol), photosynthesis (as carotenoids), and growth regulation (as gibberellins). Isoprenoids/terpenoids may also be used in pharmaceuticals, fragrances, and biofuels due to their diverse chemical properties and biological activities.

With continued reference to FIG. 1, it is worth noting that this step 105 of method 100 may not be limited to production of isoprene only. As a nonlimiting example, the precursor for producing isoprene, such as without limitation IPP and/or DMAPP, may also be a building block for other isoprenoids, terpenes, or terpenoids; therefore, an accelerated biosynthesis of isoprene may contribute to an increase in yield for producing these isoprenoids, terpenes, or terpenoids. Specifically, in some cases, products of the MVA pathway such as DMAPP and IPP may be converted to geranyl diphosphate (GPP), which may be further converted to monoterpenes, etc. In some cases, products of the MVA pathway such as DMAPP and IPP may be converted to farnesyl diphosphate (FPP), which may be further converted to sesquiterpenes, triterpenes, and/or carotenoids, among others. A person of ordinary skill in the art, upon reviewing the entirety of this disclosure, will be able to recognize how the invention described herein may be extended to other related applications beyond isoprene synthesis.

With continued reference to FIG. 1, in one or more embodiments, at step 110, method may further include purifying, using a first purification apparatus, first product to isolate isoprene. In some cases, first purification apparatus may include an absorbing and stripping apparatus. An absorbing and stripping apparatus may include one or more absorbing and stripping columns. For the purposes of this disclosure, an “absorbing and stripping column” is an apparatus, usually a vertical vessel, that is often used in chemical engineering processes to facilitate a transfer of one or more specific components between gas and liquid phases. Additional details regarding absorption/stripping apparatus will be provided below in this disclosure.

With continued reference to FIG. 1, at step 115, method 100 further includes dimerizing isoprene, using a transition metal-based catalyst, to form a second product including dimethylcyclooctadiene. It is worth noting that different types of transition metal-based catalysts (e.g., with different types of metal centers or the like) may have different suitable temperature windows in order to perform their respective catalytic functions. As a result, the temperature or temperature range to be used for step 115 may vary from case to case, depending on the exact type of transition metal-based catalyst being used. In one or more embodiments, for some transition metal-based catalysts, step 115 may be performed at a temperature from 5° C. to 60° C. In some cases, when using certain transition metal-based catalyst, step 115 may progress very slowly at or below 5° C. Additionally, and/or alternatively, in some cases, some transition-metal catalysts may deactivate at a temperature above 60° C. As nonlimiting examples, step 115 may be performed at a temperature between 5° C. and 10° C., between 10° C. and 15° C., between 15° C. and 20° C., between 20° C. and 25° C., between 25° C. and 30° C., between 30° C. and 35° C., between 35° C. and 40° C., between 40° C. and 45° C., between 45° C. and 50° C., between 50° C. and 55° C., or between 55° C. and 60° C. As further nonlimiting examples, step 115 may be performed at approximately 5° C., approximately 10° C., approximately 15° C., approximately 20° C., approximately 25° C., approximately 30° C., approximately 35° C., approximately 40° C., approximately 45° C., approximately 50° C., approximately 55° C., or approximately 60° C. It is worth noting that step 115 may preferably take place within a temperature range between 40° C. and 45° C., and, in some cases, the choice of temperature may impact the distribution of dimethylcyclooctadiene isomers in second product. Additional details pertaining to dimethylcyclooctadiene isomers will be provided below in this disclosure.

With continued reference to FIG. 1, in one or more embodiments, for some transition-metal-based catalysts, step 115 may be performed under a temperature at or above 60° C. In one or more embodiments, step 115 may be performed at a temperature below 125° C. in order to suppress competing side reactions, as described in further detail below. As nonlimiting examples, step 115 may be performed at a temperature between 60° C. and 70° C., between 70° C. and 80° C., between 80° C. and 90° C., between 90° C. and 100° C., between 100° C. and 110° C., between 110° C. and 120° C., or between 120° C. and 125° C. As further nonlimiting examples, step 115 may be performed at approximately 70° C., approximately 80° C., approximately 90° C., approximately 100° C., approximately 110° C., or approximately 120° C.

With continued reference to FIG. 1, at step 115, dimerization of isoprene may be performed at a pressure from ambient pressure (0 psig) to 200 psig. The choice of pressure under which to perform step 115 may depend on the pressure rating of a reactor. As nonlimiting examples, step 115 may be performed under a pressure between 0 psig and 10 psig, between 10 psig and 20 psig, between 20 psig and 30 psig, between 30 psig and 40 psig, between 40 psig and 50 psig, between 50 psig and 60 psig, between 60 psig and 70 psig, between 70 psig and 80 psig, between 80 psig and 90 psig, between 90 psig and 100 psig, between 100 psig and 110 psig, between 110 psig and 120 psig, between 120 psig and 130 psig, between 130 psig and 140 psig, between 140 psig and 150 psig, between 150 psig and 160 psig, between 160 psig and 170 psig, between 170 psig and 180 psig, between 180 psig and 190 psig, or between 190 psig and 200 psig. As nonlimiting examples, step 115 may be performed under a pressure of approximately 0 psig, approximately 10 psig, approximately 20 psig, approximately 30 psig, approximately 40 psig, approximately 50 psig, approximately 60 psig, approximately 70 psig, approximately 80 psig, approximately 90 psig, approximately 100 psig, approximately 110 psig, approximately 120 psig, approximately 130 psig, approximately 140 psig, approximately 150 psig, approximately 160 psig, approximately 170 psig, approximately 180 psig, approximately 190 psig, or approximately 200 psig. In some cases, step 115 may preferably proceed at a pressure between 30 psig and 40 psig. In some cases, such as in a streamlined continuous reactor, step 115 may preferably proceed at a pressure between 80 psig and 100 psig.

With continued reference to FIG. 1, for the purposes of this disclosure, a “transition metal-based catalyst” is a catalyst that contains at least a transition-metal element. Due to their incompletely filled d or f electron subshells, atoms or ions of transition metals often exhibit catalytic activities not shown in main-group metal elements. In one or more embodiments, transition metal-based catalyst may include a coordination complex, wherein one or more ions or molecules called ligands are bonded to and stabilizing a central transition-metal atom or ion via coordinate covalent bonds. In one or more embodiments, transition metal-based catalyst may include an iron (Fe)-based catalyst (abbreviated as [Fe]), a cobalt (Co)-based catalyst, a nickel (Ni)-based catalyst, or a manganese (Mn)-based catalyst, among others. As a nonlimiting example, transition metal-based catalyst may include a Cp*Ru(η⁴-isoprene) halide catalyst (Cp*=η⁵—C₅Me₅) combined with silver trifluoromethanesulfonate/silver triflate (i.e., CF₃SO₃Ag or AgOTf). As another nonlimiting example, transition metal-based catalyst may include [(^{Me}PI)FeCl(μ-Cl)]₂ (^{Me}PI=[2-(2,6-(CH₃)₂—C₆H₃—N═C(CH₃))—C₄H₅N]).

With continued reference to FIG. 1, in one or more embodiments, transition metal-based catalyst may include one or more triphenylphosphine (TPP)-based ligands. For the purposes of this disclosure, a “triphenylphosphine (TPP)-based ligand” is a molecule that includes a triphenylphosphine structure. For the purposes of this disclosure, triphenylphosphine (TPP) is an organophosphorus compound with the chemical formula C₁₈H₁₅P, characterized by a phosphorus atom bonded to three phenyl (—C₆H₅) groups. TPP may be utilized as a versatile reagent in organic synthesis, particularly in the Wittig reaction for the formation of alkenes and in transition metal-catalyzed processes such as hydrogenation, hydroformylation, and cross-coupling reactions. Due to the lone pair located on the phosphorus atom, TPP is able to function as a ligand and stabilize various metal complexes, which makes it suitable for facilitating many chemical transformations in pharmaceuticals, agrochemicals, and fine chemicals production. As non-limiting examples, a TPP-based ligand may include an unsubstituted TPP. As another nonlimiting example, a TPP-based ligand may include a substituted TPP, wherein one or more substituents may be positioned at the ortho, meta, and/or para positions of the phenyl group, thereby imparting steric and/or electronic effects to the substituted TPP. Nonlimiting examples of substituents include methyl (—CH₃), ethyl (—C₂H₅), propyl (—C₃H₇), isopropyl (—CH(CH₃)₂), butyl (—C₄H₉), hydroxyl (—OH), amino (—NH₂), nitro (—NO₂), fluorine (—F), chlorine (—C₁), bromine (—Br), iodine (—I), phenyl (—C₆H₅), cyano (—C≡N), carboxyl (—COOH), aldehyde (—CHO), keto (—C—O), methoxy (—OCH₃), sulfhydryl (—SH), trifluoromethyl (—CF₃), acetyl (—COCH₃), vinyl (—CH═CH₂), allyl (—CH₂CH—CH₂), benzyl (—CH₂C₆H₅), isobutyl (—CH₂CH(CH₃) 2), sec-butyl (—CH(CH₃) CH₂CH₃), tert-butyl (—C(CH₃) 3), ethoxy (—OCH₂CH₃), formyl (—CHO), isocyanate (—NCO), azido (—N₃), nitroso (—NO), oxo (—O), thio (—S), and carboxamide (—CONH₂), among others. In some cases, a combination of two or more triphenylphosphine (TPP)-based ligands may be present in a single transition metal-based catalyst in order to fine-tune its reactivity and catalytic function. In some cases, the triphenylphosphine (TPP)-based ligands may be unsymmetrical in a single transition metal-based catalyst in order to fine-tune its reactivity and catalytic function. A person of ordinary skill in the art, upon reviewing the entirety of this disclosure, will be able to recognize how TPP may be incorporated in a transition metal-based catalyst.

With continued reference to FIG. 1, in one or more embodiments, transition metal-based catalyst may include one or more trialkylphosphine ligands and/or one or more derivatives thereof. For the purposes of this disclosure, a “trialkylphosphine ligand” is an organophosphorus compound with the chemical formula PR₃, wherein R is an alkyl group that contains only C—C and C—H bonds. An alkyl group may contain a linear alkyl group, a branched alkyl group, and/or a cycloalkyl group. Trialkylphosphines are generally electron-donating ligands, and such electron-donating effect may impact the reactivity, stability, and selectivity of a transition metal-based catalyst. The steric and electronic properties of trialkylphosphines may be tuned by varying one or more alkyl groups, such as without limitation by changing their length and/or branching. Nonlimiting examples of trialkylphosphines may include trimethylphosphine (PMe₃), where all three alkyl groups are methyl groups; triethylphosphine (PEt₃), where all three alkyl groups are ethyl groups; tripropylphosphine (PPr₃), where all three alkyl groups are propyl groups; triisopropylphosphine (PiPr₃), where all three alkyl groups are isopropyl groups; tributylphosphine (P(n-Bu)₃), where all three alkyl groups are n-butyl groups; triisobutylphosphine (PiBu₃), where all three alkyl groups are isobutyl groups; tri-tert-butylphosphine (P(t-Bu)₃), where all three alkyl groups are tert-butyl groups; tricyclohexylphosphine (PCy₃), where all three alkyl groups are cyclohexyl groups; tri-n-octylphosphine (P(n-Oct)₃), where all three alkyl groups are n-octyl groups; or the like. In some cases, the three alkyl groups of a trialkylphosphine ligand may not be the same. Alternatively, and/or additionally, the steric and electronic properties of an unsubstituted trialkylphosphine may be fine-tuned by replacing one or more H atoms in one or more alkyl groups with one or more substituents, consistent with details described above.

With continued reference to FIG. 1, similarly, in one or more embodiments, transition metal-based catalyst may include one or more trialkylphosphite ligands and/or one or more derivatives thereof, consistent with details described above. For the purposes of this disclosure, a “trialkylphosphite ligand” is an organophosphorus compound with the chemical formula POR₃, wherein R is an alkyl group that contains only C—C and C—H bonds. A trialkylphophite ligand may be used to coordinate to metal centers in various catalytic or coordination complexes, serving to modify the reactivity, stability, or electronic properties of the metal centers. Trialkylphosphite ligands are commonly used in catalysis, where they influence the efficiency and selectivity of chemical reactions. The alkyl group within a trialkylphosphite ligand may include without limitation any type of alkyl group or combination thereof described above pertaining to trialkylphosphine ligands.

With continued reference to FIG. 1, in one or more embodiments, transition metal-based catalyst may include one or more organophosphate ligands or one or more derivatives thereof. For the purposes of this disclosure, an “organophosphate” is a type of organic compound, typically in the form of a phosphate ester, that contains a phosphorus bonded to three carbon atoms via three oxygen atoms. The general structure of an organophosphate is (RO)₃P═O, wherein the R groups may include various organic moieties such as without limitation alkyl, aryl, or other substituents. As a ligand, an organophosphate may coordinate to metal centers through the oxygen atom within the P═O bond. Due to the large difference in electronegativity between phosphorus and oxygen, the P═O bond is highly polarized, with the oxygen being partially negative and the phosphorus being partially positive, allowing organophosphates to act as electron donors to metal centers. Therefore, organophosphates may form stable complexes with metals, particularly those that are electron-deficient and capable of accepting electron density from a ligand.

With continued reference to FIG. 1, in one or more embodiments, transition metal-based catalyst may include a malononitrile ligand. For the purposes of this disclosure, “malononitrile” is an organic compound with the chemical formula CH₂(CN)₂. It includes a methylene group (—CH₂—) flanked by two cyano groups (—C≡N) and is highly reactive. Malononitrile has a molar mass of 66.06 g/mol and is a colorless solid that is soluble in water and polar organic solvents. As a ligand, malononitrile may coordinate to a metal center through its nitrile/cyano groups. The electron-withdrawing nature of the cyano groups makes malononitrile a π-acceptor ligand and renders the central methylene hydrogen atoms relatively acidic. Specifically, as a π-acceptor ligand, malononitrile may be capable of stabilizing low oxidation state metals via electron back-donation from the metal into the low-lying π* orbitals of its nitrile groups. Additionally, and/or alternatively, in some cases, malononitrile may act as a sigma donor. Additionally, and/or alternatively, in some cases, malononitrile may act as a proton donor (H). The pKₐ of malononitrile is approximately 11.2. For reference, the pKₐs of HCO₃⁻ and HPO₄⁻ are 10.3 and 12.3, respectively. As a ligand, malononitrile may form stable complexes with metals such as without limitation palladium, platinum, and copper, among others. These complexes may be used to catalyze reactions such as without limitation cross-coupling reactions.

With continued reference to FIG. 1, in some cases, transition metal-based catalyst may include two or more types of ligands described above at the same time. As a nonlimiting example, transition metal-based catalyst may include two or more types of TPP-based ligands. As another nonlimiting example, transition metal-based catalyst may include two or more types of trialkylphosphine ligands (or derivatives thereof). As another nonlimiting example, transition metal-based catalyst may include two or more organophosphate ligands (or derivatives thereof) at the same time. As another nonlimiting example, transition metal-based catalyst may include one or more trialkylphosphine ligands (or derivatives thereof) and one or more TPP-based ligands at the same time. As another nonlimiting example, transition metal-based catalyst may include a combination of one or more TPP-based ligands, one or more trialkylphosphine ligands, one or more organophosphate ligands, and/or one or more malononitrile ligands.

With continued reference to FIG. 1, in one or more embodiments, transition metal-based catalyst may include an organometallic catalyst. For the purposes of this disclosure, an “organometallic catalyst” is a catalyst based on an organometallic compound. For the purposes of this disclosure, an “organometallic compound” is a compound containing at least one bond between a carbon atom of an organic molecule/ion and a metal atom/ion. Organometallic compounds are often characterized by their unique reactivity and are extensively used in catalysis, material science, and organic synthesis. As nonlimiting examples, an organometallic compound may be able to participate in steps such as oxidative addition, elimination such as reductive elimination, insertion/migratory insertion, transmetalation, hydride transfer, ligand exchange, among others. A person of ordinary skill in the art, upon reviewing the entirety of this disclosure, will be able to recognize how an organometallic catalyst may be used for one or more aspects of method 100.

With continued reference to FIG. 1, for the purposes of this disclosure, “dimethylcyclooctadiene” is a hydrocarbon molecule with a formula of C₁₀H₁₆ that contains two methyl groups attached to an eight-membered ring, within two C═C bonds disposed within the eight-membered ring. Dimethylcyclooctadiene has a molar mass of 136.23 g/mol. In one or more embodiments, dimethylcyclooctadiene may include 1,5-dimethyl-1,5-cyclooctadiene, 1,6-dimethyl-1,5-cyclooctadiene, 2,6-dimethyl-1,3-cyclooctadiene, 2,6-dimethyl-1,4-cyclooctadiene, 3,7-dimethyl-1,5-cyclooctadiene, 3,7-dimethyl-1,3-cyclooctadiene, and 3,6-dimethyl-1,3-cyclooctadiene, among other potential variations thereof due to rearrangements. As a nonlimiting example, 3,7-dimethyl-1,5-cyclooctadiene has a boiling point of 182.7° C., a density of 0.860 g/mL, a vapor pressure of 1.09 Torr at 25° C., a molar heat of combustion of 1490 KJ/mol, a lower heating value of 134.0 kBtu/gallon, a higher heating value of 141.4 kBtu/gallon, and an octane rating of 95.

With continued reference to FIG. 1, in one or more embodiments, second product may include at least a byproduct. In some cases, such byproduct may include at least a Diels-Alder [4+2] cycloaddition product. Nonlimiting examples of such byproduct may include 1-methyl-4-(prop-1-en-2-yl)cyclohex-1-ene, 1-methyl-5-(prop-1-en-2-yl)cyclohex-1-ene, 1,4-dimethyl-4-vinylcyclohexene, 2,4-dimethyl-4-vinylcyclohexene, among other potential variations thereof due to rearrangements. In some cases, at around or above 125° C., the Diels-Alder [4+2] cycloaddition product may dominate the product distribution of step 115 and outcompete the dimerization of isoprene. Therefore, in order to improve the yield of dimethylcyclooctadiene, it is generally preferrable to perform step 115 of method 100 at a temperature below 125° C.

With continued reference to FIG. 1, in one or more embodiments, at step 120, method 100 may further include purifying, using a second purification apparatus, second product to isolate dimethylcyclooctadiene from at least a byproduct, consistent with details described above. It is worth noting that this step may be optional, and certain byproducts may be used for hydrogenation in the following steps to form hydrogenated byproducts. Additional details will be provided below.

With continued reference to FIG. 1, in some cases, second purification apparatus may include a distillation apparatus. For the purposes of this disclosure, a “distillation apparatus” is a system designed to separate components of a liquid mixture based on differences in their boiling points. Upon heating a mixture, one or more volatile components therein with relatively lower boiling points are vaporized and then condensed/collected separately. A basic distillation apparatus typically includes components such as a boiling flask, a condenser, a distillation column, and a receiving flask, though more intricate variations are often used for scalable production in a manufacturing context. Nonlimiting examples of a distillation apparatus may include simple distillation apparatus, fractional distillation columns, vacuum distillation systems, steam distillation setups, and azeotropic distillation apparatuses, among others. A person of ordinary skill in the art, upon reviewing the entirety of this application, will be able to recognize how a distillation apparatus may be implemented for method 100. Additionally, and/or alternatively, second purification apparatus may include one or more filtration apparatus, the details of which will be described in detail below when discussing steps 130 and 135.

With continued reference to FIG. 1, at step 125, method 100 further includes hydrogenating, using a hydrogenation catalyst, dimethylcyclooctadiene to form a third product. In one or more embodiments, at a relatively small scale, step 125 may be performed at ambient conditions such as at a temperature between 20° C. and 25° C. (i.e., room temperature) and at a pressure of approximately 1 atm. In some cases, at a relatively small scale, step 115 may be performed in a glass reactor, using a room-temperature water bath as a heat sink, by applying a balloon pressure of approximately 0.2 psig H₂ gas. For the purposes of this disclosure, a “balloon pressure” is a pressure exerted by a gas-filled balloon connected to a reaction vessel. This setup is often used in organic synthesis to maintain a consistent, low-pressure supply of a gas, such as without limitation hydrogen gas, nitrogen gas, or oxygen gas, over the course of a chemical reaction. In one or more embodiments, at a relatively large scale, step 125 may be performed at an elevated temperature and/or pressure. As a nonlimiting example, step 125 may be performed in a reactor with a capacity of approximately 1650 gallons, at a temperature of approximately 300° C. and a pressure below 1000 psig. As another nonlimiting example, step 125 may be performed at a temperature between 150° C. and 200° C. (for example, between 150° C. and 160° C., between 160° C. and 170° C., between 170° C. and 180° C., between 180° C. and 190° C., or between 190° C. and 200° C.). As another nonlimiting example, step 125 may be performed at a pressure below 400 psig (for example, below 400 psig, below 350 psig, below 300 psig, below 250 psig, below 200 psig, below 150 psig, below 100 psig, below 50 psig, or the like). It is worth noting that high pressures are more commonly applied to small-scale reactors than to large-scale reactors, due to their different manufacturing constraints. Under the same pressure rating, as the size of a reactor increases, the reactor may become more and more challenging to manufacture and/or maintain. As a result, in a scaled-up reactor, a use of high pressure may result in a higher cost.

With continued reference to FIG. 1, for the purposes of this disclosure, a “hydrogenation catalyst” is a catalyst capable of adding a plurality of hydrogen atoms across an unsaturated chemical bond in a molecule or ion, thereby increasing the degree of saturation in the molecule or ion. Unsaturated chemical bond may include a double bond such as a C═C bond or a triple bond such as a C═C bond. In one or more embodiments, hydrogenation catalyst may include without limitation an iron (Fe)-based catalyst, a magnesium (Mg)-based catalyst, a ruthenium (Ru)-based catalyst, an aluminum (Al)-based catalyst, a nickel (Ni)-based catalyst including a rhenium-nickel (Re—Ni) alloy, a nickel-aluminum (Ni—Al) alloy, Raney nickel, Ni supported on silica-alumina (SiO₂—Al₂O₃), among others, a chromium (Cr)-based catalyst, a platinum(Pt)-based catalyst including platinum(IV) oxide (PtO₂) (which is also known as platinum(IV) oxide hydrate, PtO₂·H₂O, platinum dioxide, or Adam's catalyst), and/or a palladium (Pd)-based catalyst including Pd black, Pd supported on carbon (Pd/C), Pd supported on CaCO₃ (Pd/CaCO₃), Pd supported on BaSO₄ (Pd/BaSO₄), Pd supported on SiO₂ (Pd/SiO₂), and Pd supported on CaCO₃ (Pd/Al₂O₃), among others. A person of ordinary skill in the art, upon reviewing the entirety of this disclosure, will be able to recognize proper means to implement a hydrogenation catalyst in method 100.

With continued reference to FIG. 1, third product includes dimethylcyclooctane. For the purposes of this disclosure, dimethylcyclooctane is a hydrocarbon molecule with a formula C₁₀H₂₀ that contains two methyl groups attached to an eight-membered ring. Dimethylcyclooctane has a molar mass of 140.27 g/mol. Dimethylcyclooctane is expected to have favorable fuel properties due to its cyclic structure and/or chain branching. Specifically, the cyclic structure and ring strain of dimethylcyclooctane may afford a gravimetric/volumetric net heat of combustion that is 2.4%/9.2% higher, respectively, than a conventional jet fuel. In addition, the presence of methyl substituents at two sites results in a −20° C. kinematic viscosity of 4.17 mm²·s⁻¹, which is 48% lower than the maximum allowed value for conventional jet fuel. Dimethylcyclooctane is a 100% sustainable aviation fuel.

With continued reference to FIG. 1, in one or more embodiments, dimethylcyclooctane may include 1,4-dimethylcyclooctane (1,4-DMCO), i.e., cis-1,4-dimethylcyclooctane and trans-1,4-dimethylcyclooctane. In one or more embodiments, dimethylcyclooctane may include 1,5-dimethylcyclooctane (1,5-DMCO), i.e., cis-1,5-dimethylcyclooctane or trans-1,5-dimethylcyclooctane. These four isomers of dimethylcyclooctane may account for more than 99% of the product. As a nonlimiting example, 1,5-dimethylcyclooctane has a boiling point of 148.5° C., a density of 0.800 g/mL, a vapor pressure of 1.39 Torr at 25° C., a molar heat of combustion of 1560 KJ/mol, a lower heating value of 125.4 kBtu/gallon, a higher heating value of 133.8 kBtu/gallon, and an octane rating of 85. Other non-dominant products may include without limitation 1,1-dimethylcyclooctane, cis-1,2-dimethylcyclooctane, trans-1,2-dimethylcyclooctane, and cis-1,3-dimethylcyclooctane, and trans-1,3-dimethylcyclooctane. These non-dominant products are likely hydrogenated products of minor dimethylcyclooctadiene isomers described above.

With continued reference to FIG. 1, in one or more embodiments, byproducts from step 115, such as Diels-Alder [4+2] cycloaddition products described above, may constitute a fraction of second product. Accordingly, these byproducts may be hydrogenated as part of step 125, consistent with details described above, to form their respective, hydrogenated byproducts as part of third product. These hydrogenated byproducts may also be used as aviation fuel. In some cases with more stringent requirements, these hydrogenated byproducts may be distilled from third product and therefore eliminated, consistent with details described in this disclosure.

With continued reference to FIG. 1, exemplary reactions pertaining to method 100 are summarized in Scheme 1 below, with two major dimethylcyclooctadiene intermediates, four possible byproducts, two major dimethylcyclooctane products, and four possible hydrogenated byproducts included.

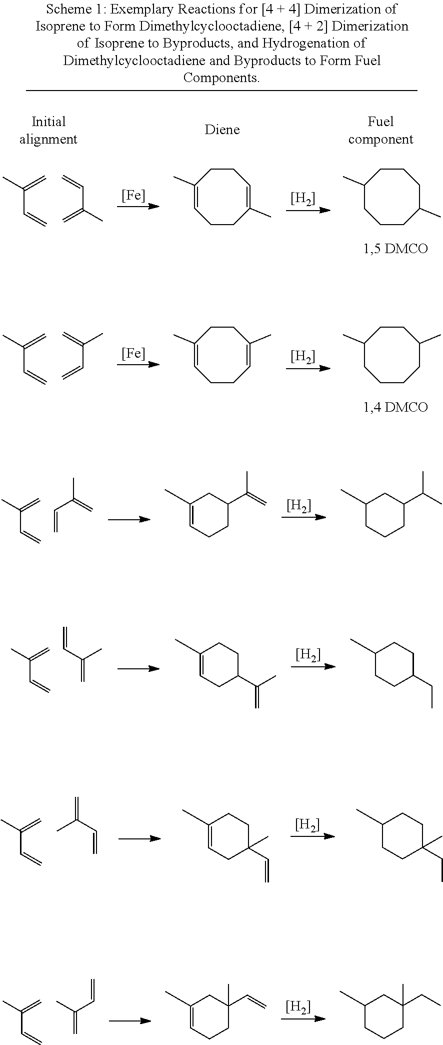

With continued reference to FIG. 1, method 100 may include one or more filtration steps to remove impurities such as metal impurities. It is worth noting that these filtration steps may be optional and, in some cases, could be replaced with a distillation step, consistent with details described above in this disclosure. In one or more embodiments, at step 130, method 100 may further include filtrating third product, using a first filter, to form a first purified product. First filter may include any type of filter deemed suitable by a person of ordinary skill in the art upon reviewing the entirety of this disclosure, such as without limitation sieve filters, membrane filters, barrier filters, clay filters, filter aids, or the like. Additionally, and/or alternatively, one or more settling tanks may be used in addition to or instead of first filter. In some cases, first filter may include a pore size between 100 nanometers and 10 micrometers. As nonlimiting examples, first filter may include a pore size between 100 nanometers and 200 nanometers, between 200 nanometers and 300 nanometers, between 300 nanometers and 400 nanometers, between 400 nanometers and 500 nanometers, between 500 nm and 1 micrometer, between 1 micrometer and 2 micrometers, between 2 micrometers and 5 micrometers, or between 5 micrometers and 10 micrometers. As a nonlimiting example, first filter may preferably include a pore size between 500 nanometers and 1 micrometer. In some cases, first filter may include a pore size of approximately 100 nanometers, approximately 200 nanometers, approximately 300 nanometers, approximately 400 nanometers, approximately 500 nanometers, approximately 1 micrometer, approximately 2 micrometers, approximately 5 micrometers, or approximately 10 micrometers.

With continued reference to FIG. 1, in one or more embodiments, at step 135, method 100 may further include filtrating first purified product, using a second filter, to form a second purified product. In some cases, second filter may include a clay filter. For the purposes of this disclosure, a “clay filter” is a filtration device utilizing porous clay materials to remove impurities, contaminants, or particulates from liquids or gases. Known for its high adsorption capacity and chemical inertness, a clay filter is commonly used in industrial fluid purification, potable water treatment, and the food and beverage industry. Nonlimiting examples of a clay filter may include activated clay filters for decolorizing and purifying oils, bentonite clay filters for removing heavy metals and organic compounds in water treatment, and kaolin clay filters for clarifying juices and wines. These clay filters may provide efficient, cost-effective solutions for applications that require high purity and cleanliness. It is worth noting that step 130 and step 135 may in some cases change orders with each other, depending on the exact use case.

With continued reference to FIG. 1, in some cases, step 130 and/or step 135 may include filtering third product, first purified product, and/or second purified product using a filter aid. For the purposes of this disclosure, a “filter aid” is a substance added to a liquid or slurry to improve the efficiency and effectiveness of a filtration process. By creating a porous filter cake, a filter aid may allow a liquid to pass through a filter more easily (i.e., with an increased flow rate), reducing the pressure drop across the filter and thereby speeding up the filtration process. Filter aids may prevent fine particles from clogging the pores of a filter medium, which may otherwise reduce the efficiency of filtration and require frequent cleaning or replacement of the filter. Filter aids may also help achieve a clearer filtrate by trapping very fine or colloidal particles that would otherwise pass through a filter medium. A filter aid may work by forming a precoat layer on a filter medium before a slurry is introduced; this layer may act as a primary filtration medium. A filter aid may alternatively work by being mixed with a slurry, as a body feed, to prevent fine particles from blinding to a filter, ensuring that they remain suspended and are captured more effectively during filtration. Filter aids are typically fine, inert, porous materials. Common filter aids may include without limitation materials such as diatomaceous earth, perlite, cellulose, and activated carbon, among others. These substances are potentially used as filter aids due to their porosity, low density, and chemical inertness, which allow them to trap fine particles without reacting with a filtrate.

With continued reference to FIG. 1, it is worth noting that any filtration material, apparatus, or technique described above pertaining to steps 130 and 135 may also apply to step 120 as part of second purification apparatus without limitation.

With continued reference to FIG. 1, it is also worth noting that method 100 described herein may not be limited to production of dimethylcyclooctane only. Instead, method 100 may be applied to produce other 10-carbon cycloalkanes, olefines, cycloparaffins, and/or cyclodienes, as recognized by a person of ordinary skill in the art, upon reviewing the entirety of this disclosure.

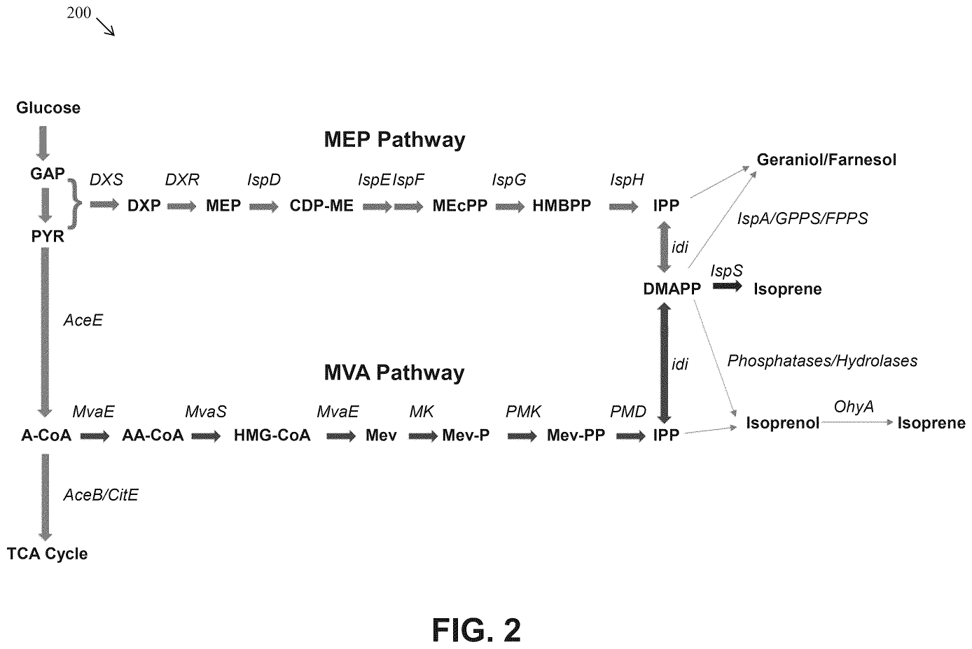

Referring now to FIG. 2, scheme 200 illustrates several metabolic pathways pertaining to biosynthesis of isoprene, highlighting the substrate, product, and/or enzyme (catalyst) associated with each step. Scheme 200 includes part of the central metabolic pathway, i.e., the cellular respiration pathway, wherein glucose is broken down to feed the tricarboxylic acid (TCA) cycle. Conversion of glucose involves several key steps. Initially, glucose (C₆H₁₂O₆) undergoes glycolysis by converting to two molecules of glyceraldehyde 3-phosphate (GAP). Such conversion is catalyzed by enzymes including hexokinase, phosphofructokinase, and aldolase. GAP is then further processed to form pyruvate (C₃H₃O₃⁻), producing a net gain of 2 ATP and 1 NADH. Such conversion is catalyzed by enzymes such as glyceraldehyde 3-phosphate dehydrogenase and pyruvate kinase. Pyruvate is further converted to acetyl coenzyme A (A-CoA), releasing NADH and CO₂. Such conversion is catalyzed by pyruvate dehydrogenase (PDH), which may include pyruvate dehydrogenase E1 component (encoded by gene AceE). A-CoA then enters the TCA cycle (not shown) and participates in reactions catalyzed by enzymes such as without limitation malate synthase (AceB) or ATP citrate lyase (CitE). It is worth noting that glucose is not the only viable carbon source for the invention described in this disclosure; alternatives such as sucrose, biomass, glycerol, ethanol, plant oils, or the like, may be used instead upon strategic engineering of metabolic pathways.

With continued reference to FIG. 2, scheme 200 includes methylerythritol phosphate (MEP) and mevalonate (MVA) pathways for isoprene production. Both the MEP and the MVA pathways are connected to the central metabolic pathway and may lead to production of isoprene, consistent with details described above. For the purposes of this disclosure, the methylerythritol phosphate (MEP) pathway, also known as the non-mevalonate pathway, is a metabolic route for isoprenoid biosynthesis in bacteria, algae, and/or plant plastids. It begins with the formation of 1-deoxy-D-xylulose-5-phosphate (DXP) from pyruvate and glyceraldehyde-3-phosphate (GAP), which is catalyzed by 1-deoxy-D-xylulose-5-phosphate synthase (DXS). This step is one of the rate-determining steps of the MEP pathway. DXP is then converted to 2-(′-methyl-D-erythritol-4-phosphate (MEP), which is catalyzed by 1-deoxy-D-xylulose 5-phosphate reductase (DXR). MEP is then converted to 4-diphosphocytidyl-2-(′-methyl-D-erythritol (CDP-ME), which is catalyzed by 4-diphosphocytidyl-2-(′-methyl-D-erythritol synthase (IspD). CDP-ME is further converted via two steps to 2-(′-methyl-D-erythritol-2,4-cyclodiphosphate (MEcPP), which is catalyzed by 4-diphosphocytidyl-2-(′-methyl-Derythritol kinase (IspE) and 2-(′-methyl-D-erythritol 2,4-cyclodiphosphate synthase (IspF). MEcPP is then reduced to 4-hydroxy-3-methylbut-2-enyl diphosphate (HMBPP), which is catalyzed by (E)-4-Hydroxy-3-methylbut-2-enyl pyrophosphate synthase (IspG). HMBPP is then converted to isopentenyl diphosphate (IPP) and dimethylallyl diphosphate (DMAPP), which is catalyzed by (E)-4-Hydroxy-3-methylbut-2-enyl pyrophosphate reductase (IspH or HMBPP reductase). IPP and DMAPP may establish a chemical equilibrium and interconvert with one another, which is catalyzed by isopentenyl diphosphate isomerase (idi). DMAPP may be converted to isoprene, which is catalyzed by isoprene synthase (IspS). IspS may be derived from plants, as many plants such as kudzu (the Asian vine, Pueraria montana), aspen (Populus tremuloides), and poplar (Populus), among others, may naturally produce isoprene by the catalytic elimination of pyrophosphate from DMAPP. Chemical structures of each reactant, intermediate, and product of the MEP pathway are illustrated in Scheme 2 below:

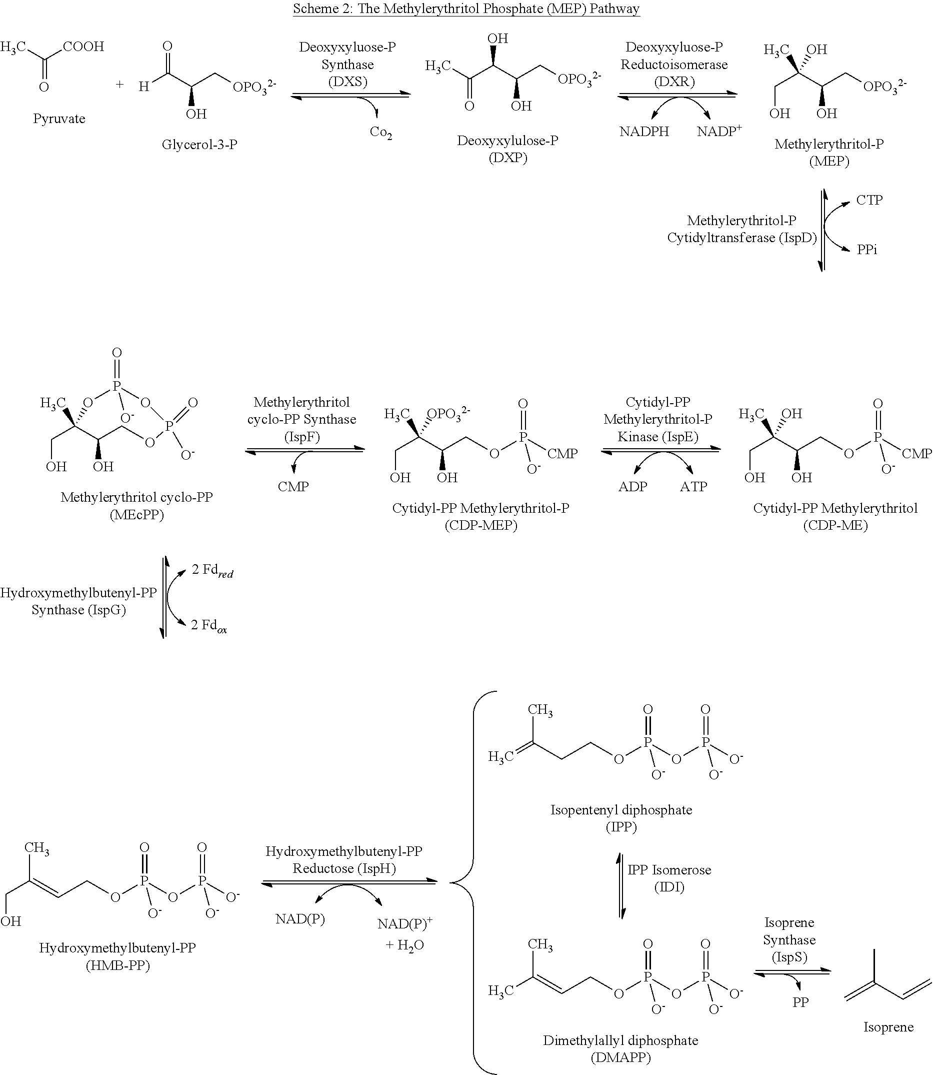

With continued reference to FIG. 2, for the purposes of this disclosure, the mevalonate (MVA) pathway is another metabolic route for isoprenoid biosynthesis, found in archaea, fungi, animals, and some bacteria. The MVA pathway starts with the conversion of A-CoA to acetoacetyl-coenzyme A (AA-CoA), which is catalyzed by acetoacetyl-coenzyme A thiolase or 3-hydroxy-3-methylglutaryl-coenzyme A reductase (MvaE, which may be encoded by gene ERG10). AA-CoA is then converted to 3-hydroxy-3-methylglutaryl-CoA (HMG-COA), which is catalyzed by HMG-CoA synthase (MvaS, which may be encoded by gene ERG13). HMG-CoA is then reduced to mevalonate (Mev). This step is catalyzed by MvaE, which may be encoded by gene HMG1, HMG2, or HMG-COA reductase (HMGR). This step is the rate-determining step of the MVA pathway. Mev is phosphorylated to mevalonate-5-phosphate (Mev-P), which is catalyzed by mevalonate kinase (MK), which could be encoded by ERG12. Mev-P is phosphorylated to form mevalonate-5-diphosphate (Mev-PP), which is catalyzed by phosphomevalonate kinase (PMK), which could be encoded by ERG8. Mev-PP is subsequently decarboxylated to produce IPP, which is catalyzed by mevalonate-5-diphosphate decarboxylase or diphosphomevalonate decarboxylase (PMD), which could be encoded by MVD1. IPP and DMAPP may establish a chemical equilibrium and interconvert with one another, which is catalyzed by idi, consistent with details described above. The idi enzyme could be encoded by IDI1. DMAPP may be converted to isoprene, which is catalyzed by IspS, consistent with details described above. In some cases, the MVA pathway may be further categorized into the “upper pathway”, which converts A-CoA to MVA, and the “lower pathway”, which produces DMAPP from MVA. In some cases, the MVA pathway may be introduced (i.e., cloned) into bacteria such as E. coli (which only has a native MEP pathway) as two synthetic operons, using a combination of bacterial and yeast enzymes. As a nonlimiting example, the lower pathway operon may be integrated into the chromosome while the upper pathway operon may be expressed on one or more plasmids, optionally alongside an IspS-encoding gene or the like. Chemical structures of each reactant, intermediate, and product of the MVP pathway are illustrated in Scheme 3 below:

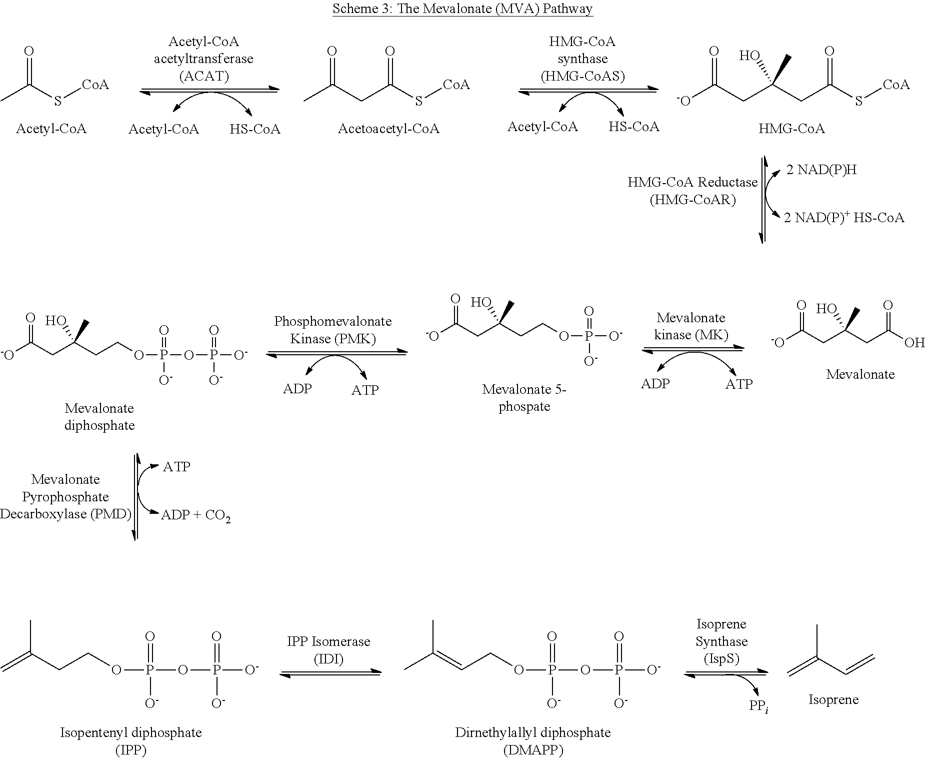

With continued reference to FIG. 2, scheme 300 includes a plurality of competing pathways that may limit the yield of isoprene production. IPP and/or DMAPP may be converted to geraniol and/or farnesol, which is catalyzed by enzymes such as without limitation geranyl diphosphate synthase (IspA), geranyl diphosphate synthase (GPPS), and/or farnesyl diphosphate synthase (FPPS). IPP and/or DMAPP may be converted to isoprenol, which is catalyzed by enzymes such as phosphatases and/or hydrolases including the Nudix hydrolase encoded by the NudB gene. As part of the central metabolic pathway, A-CoA may be converted to citrate to enter the tricarboxylic acid cycle, which is catalyzed by enzymes such as ATP citrate lyase (CitE) including AclY, AclB, and/or AclA. These pathways compete with the formation of isoprene. In some cases, to improve the yield of isoprene, byproduct isoprenol may be converted back to isoprene, which may be catalyzed by enzymes such as 3-methyl-3-buten-1-ol dehydroxylase (OhyA) or the like.

With continued reference to FIG. 2, isoprene is derived from intermediates in central metabolism. As described above, the first enzymes in both the MEP and MVA pathways use intermediates in central metabolism as substrates, namely, glyceraldehyde-3-phosphate+pyruvate or acetyl-CoA, respectively. These pathways have differing requirements for reducing power and energy. Thus, in addition to optimizing carbon flux through the MEP and/or MVA pathways, the levels of the central metabolism intermediates may also be maximized, and the reducing power/energy balance may be optimized by metabolic engineering. Additional details will be provided below in this disclosure.

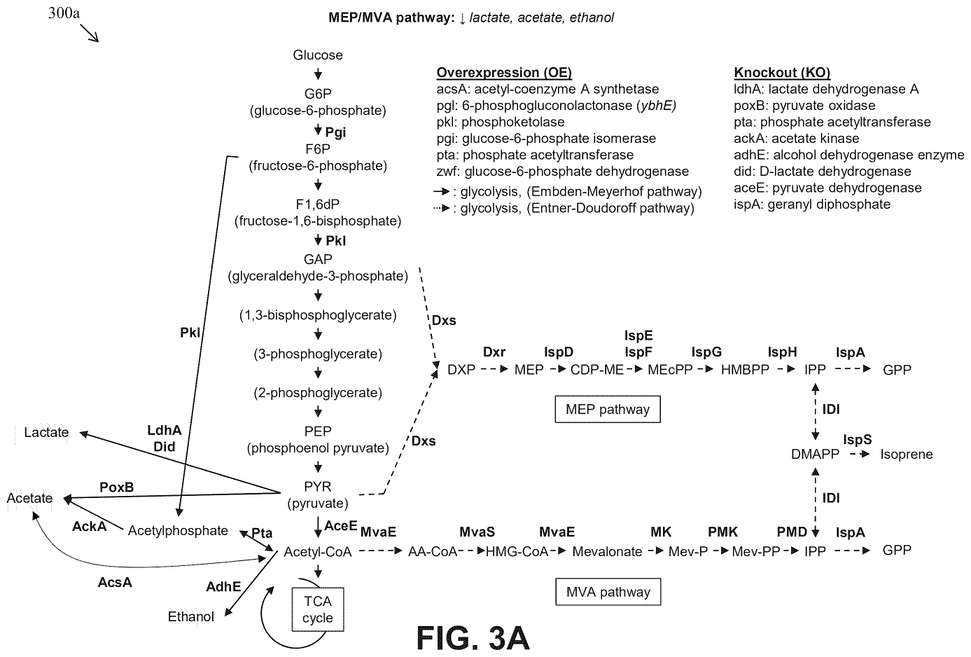

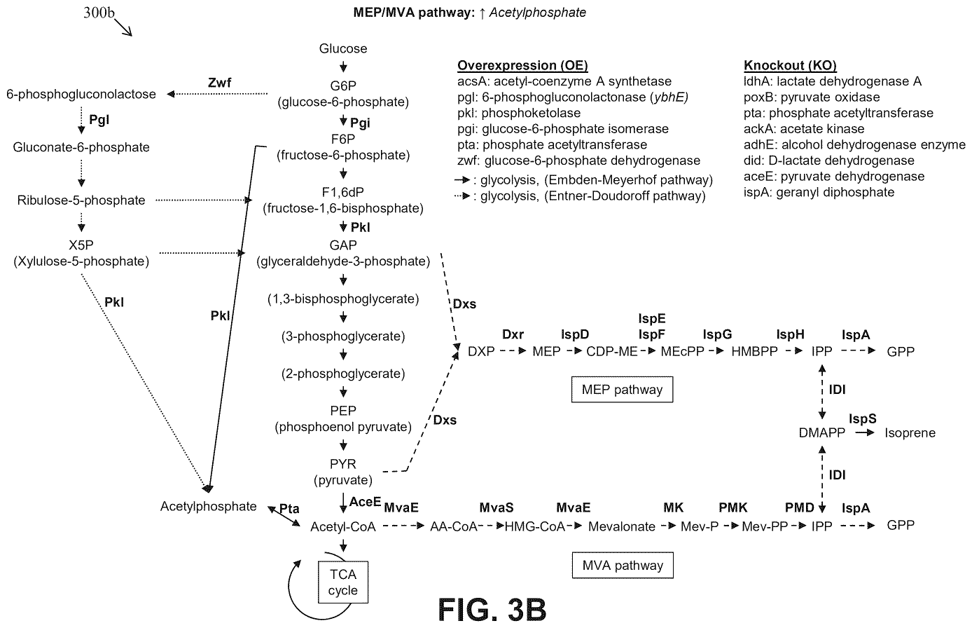

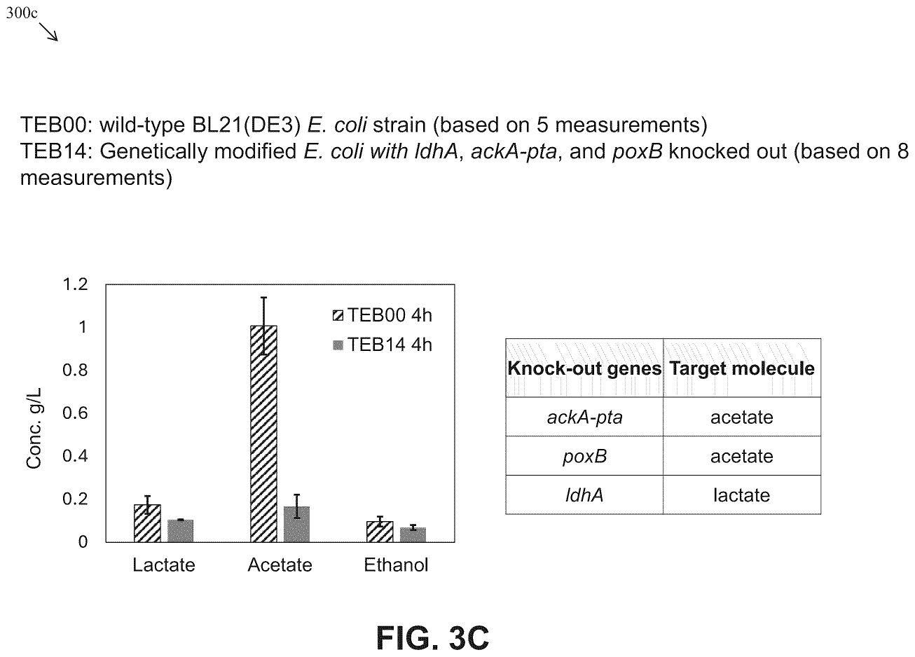

Referring now to FIGS. 3A-B, exemplary embodiments 300a-b of expanded metabolic pathways pertaining to isoprene biosynthesis are illustrated. FIGS. 3A-B include a plurality of potential gene targets. These gene targets and the activity thereof may be upregulated (for example, overexpressed), downregulated (for example, knocked down (KD)/knocked out (KO)), or otherwise modified, either individually or in combination, to increase the yield of isoprene.

With continued reference to FIGS. 3A-B, in one or more embodiments, the inherent activity of an enzyme may be improved by modifying the nucleic acid sequence that encodes the enzyme. Various techniques may be used to improve the inherent activity of an enzyme. As a nonlimiting example, directed evolution may be used to mimic natural selection in the lab by creating a large library of enzyme variants and selecting those with improved traits. As another nonlimiting example, site-directed mutagenesis may be used to introduce one or more specific mutations to enhance enzyme activity or stability. As another nonlimiting example, gene shuffling may be used to recombine segments of related genes to create new variants with enhanced properties. As another nonlimiting example, fusion proteins may be created by combining enzymes with other proteins to improve their function and/or stability. As another nonlimiting example, an enzyme such as DXS may be modified to lift one or more feedback regulations/inhibitions caused by an accumulation of its downstream products such as DMAPP, thereby improving the efficiency of isoprene synthesis. As a nonlimiting example, an enzyme such as OhyA or the like may be engineered to increase its inherent activity, thereby facilitating accumulation of isoprene while lifting the potentially toxic impact of isoprenol. As another nonlimiting example, an enzyme such MvaE or HMGR may be truncated from its naturally existing counterpart to improve its catalytic activity. As another nonlimiting example, one or more cofactors for one or more enzymes may be switched in order to improve the catalytic activity of an enzyme or enable fermentation conditions to be utilized that are more cost effective (e.g., with a lower oxygen requirement).

With continued reference to FIGS. 3A-B, in one or more embodiments, genes encoding enzymes such as 1-deoxy-D-xylulose-5-phosphate synthase (DXS), isoprene synthase (IspS), 3-hydroxy-3-methylglutaryl-coenzyme A reductase (HMG-COA reductase or HMGR), 3-methyl-3-buten-1-ol dehydroxylase (OhyA) or OhyA-like enzymes, (F)-4-hydroxy-3-methylbut-2-enyl diphosphate synthase (IspG), 1-deoxy-D-xylulose 5-phosphate reductase (DXR), (F)-4-hydroxy-3-methylbut-2-enyl diphosphate reductase (IspH or HMBPP reductase), acetyl-CoA C-acetyltransferase/acetoacetyl-coenzyme A thiolase (MvaE, encoded by gene ERG10), HMG-CoA Synthase (MvaS, encoded by gene ERG13), acylating acetaldehyde dehydrogenase (A-ALD), among others, may be modified to increase the inherent activity of the enzyme, consistent with details described in the rest of this disclosure.

With continued reference to FIGS. 3A-B, in one or more embodiments, the activity of an enzyme may be modulated by increasing or upregulating an expression of a nucleic acid construct that encodes the enzyme. In some cases, a coding sequence may be configured to upregulate one or more genes encoding enzymes such as 3-methyl-3-buten-1-ol dehydroxylase (OhyA), transcription factor (encoded by UPC2), acetyl-CoA C-acetyltransferase/acetoacetyl-coenzyme A thiolase (MvaE, encoded by gene ERG10), mevalonate kinase (MK, encoded by ERG12), HMG-COA synthase (MvaS, encoded by gene ERG13), aldehyde dehydrogenase (encoded by ALD5), acetyl-CoA synthetase (encoded by ACSA, ACS1, and/or ACS2, depending on the exact chassis used), pantothenate kinase (encoded by CAB1), dihydrolipoamide acetyltransferase component of the pyruvate dehydrogenase complex (encoded by LAT1), glucose-6-phosphate dehydrogenase (encoded by zwf), 6-phosphogluconolactonase (ybhE, encoded by pgl), phosphoketolase (encoded by pkl), glucose-6-phosphate isomerase (encoded by pgi), and phosphate acetyltransferase (encoded by pta), among others, as these genes encode enzymes that may contribute to the production and/or accumulation of isoprene. In some cases, regulation of a single gene may result in a regulation of multiple enzymes. As a nonlimiting example, when UPC2 is upregulated, ERG12 and ERG13 may also be overexpressed.

With continued reference to FIG. 3A-B, in one or more embodiments, the activity of an enzyme may be modulated by decreasing, attenuating, downregulating, or deleting the expression of a nucleic acid construct that encodes the enzyme. In some cases, a gene encoding an enzyme may be knocked down or knocked out. As nonlimiting examples, one or more genes encoding enzymes such as geranyl diphosphate synthase (IspA), farnesyl pyrophosphate synthetase (Erg 20), geranyl diphosphate synthase (GPPS), geranylgeranyl diphosphate synthase (BTS1), farnesyl diphosphate synthase (FPPS), pyruvate dehydrogenase (encoded by PDH) including pyruvate dehydrogenase E1 component (encoded by AceE), pyruvate oxidase (encoded by poxB), pyruvate decarboxylase (encoded by PDC), malate synthase (encoded by AceB or MLS1), citrate synthase (encoded by CIT1), ATP-citrate lyase (CitE) including AclY, AclB, and AclA, glycerol-producing enzymes including cGPDH and G3PP [41], ethanol-producing enzymes including invertase and zymase, alcohol dehydrogenase (encoded by ADH1), alcohol dehydrogenase enzyme (encoded by adhE), lactate-producing enzymes including lactate dehydrogenase such as lactate dehydrogenase A (encoded by IdhA) and D-lactate dehydrogenase (encoded by did), acetate-producing enzymes including acetyl coenzyme A (acetyl-CoA) hydrolases, acetate kinase (encoded by ackA), phosphatase, and/or hydrolase (for example, a hydrolase encoded by the NudB gene), among others, may be downregulated. However, it is worth noting that, in some cases, an enzyme may perform an essential function of a chassis and should not be completely knocked out or suppressed. As a nonlimiting example, FPPS encoded by ERG20 is essential to S. cerevisiae, and accordingly, specific sites within FPPS may be mutated in order to downregulate (not eliminate) its function. As another nonlimiting example, a diploid strain may be used to knockout one copy of ERG20 gene in order to downregulate the activity of the enzyme it encodes. In some cases, an enzyme may not be essential to a chassis and may be knocked out without causing adverse effects. As a nonlimiting example, genes such as MLS1 and CIT1 may be knocked out in such manner.

With continued reference to FIG. 3A-B, in some cases, multiple upregulations and downregulations may be implemented simultaneously to achieve a synergic effect. As a nonlimiting example, gene targets such as zwf, pgl, and/or pkl may be upregulated simultaneously to utilize the Entner-Doudoroff pathway in addition to the Embden-Meyerhof pathway regulated by pgi and pkl, thereby increasing the carbon flux of biosynthesis and increasing the yield of acetylphosphate (see FIG. 3A, embodiment 300ₐ). As another nonlimiting example, gene targets such as ackA-pta, poxB, and/or ldhA may be knocked down or knocked out simultaneously to suppress competing reactions that form acetate, lactate, and/or ethanol (see FIG. 3B, embodiment 300_{b}). As another nonlimiting example, simultaneous upregulation of ALD5 and downregulation of ADH1 may be combined to prevent acetyl-CoA from converting to ethanol via fermentation. Table 1 summarizes exemplary gene targets for upregulation and downregulation for S. cerevisiae and E. coli.

TABLE 1
Gene Targets for Improving Isoprene Production in S. cerevisiae and E. coli.

| | | Type of Genetic | |
|---|---|---|---|
| | | Modification | |
| | | Note: | |
| | | Knockout (KO) | |
| | | Knockdown (KD) | |
| Organism | Gene | Overexpress (OE) | Rationale |
| S. cerevisiae | ERG20: farnesyl | KD or mutate | Essential Gene; |
| | pyrophosphate synthetase | specific sites | increase DMAPP |
| S. cerevisiae | BTS1: geranylgeranyl | KO/KD | Increase DMAPP |
| | diphosphate synthase | | |
| S. cerevisiae | UPC2: transcription | OE | Increase products of |
| | factor | | MVA pathway |
| S. cerevisiae | ERG10: acetyl-CoA C- | OE | Increase products of |
| | acetyltransferase | | MVA pathway |
| S. cerevisiae | ERG12: Mevalonate | OE | Increase products of |
| | kinase | | MVA pathway |
| S. cerevisiae | ERG13: HMG-CoA | OE | Increase products of |
| | synthase | | MVA pathway |
| S. cerevisiae | ALD5: aldehyde | OE | Increase flux from |
| | dehydrogenases | | acetaldehyde to |
| | | | acetyl-CoA instead |
| | | | of ethanol |
| | | | production. Could |
| | | | also work with fed |
| | | | ethanol to push |
| | | | towards acety-CoA |
| S. cerevisiae | ADH1: alcohol | KO/KD | Increase flux from |
| | dehydrogenase | | acetaldehyde to |
| | | | acetyl-CoA instead |
| | | | of ethanol |
| | | | production. Could |
| | | | also work with fed |
| | | | ethanol to push |
| | | | towards acety-CoA |
| S. cerevisiae | ACS1 and ACS2: acetyl- | OE | Increase flux from |
| | CoA synthetase | | acetaldehyde to |
| | | | acetyl-CoA instead |
| | | | of ethanol |
| | | | production. Could |
| | | | also work with fed |
| | | | ethanol to push |
| | | | towards acety-CoA |
| S. cerevisiae | MLS1: Malate synthase | KO/KD | Increase acetyl CoA |
| | | | pool |
| S. cerevisiae | CIT1: Citrate synthase | KO/KD | Increase acetyl CoA |
| | | | pool |
| S. cerevisiae | CAB1: Pantothenate | OE | Increase acetyl CoA |
| | kinase | | pool |
| S. cerevisiae | LAT1: dihydrolipoamide | OE | Increase acetyl CoA |
| | acetyltransferase | | pool |
| | component of the | | |
| | pyruvate dehydrogenase | | |
| | complex | | |
| E. coli | zwf: glucose-6-phosphate | OE | Increase carbon flux |
| | dehydrogenase | | to acetylphosphate |
| | | | (overexpression of |
| | | | the Entner- |
| | | | Doudoroff pathway) |
| E. coli | acsA: acetyl-coenzyme A | OE | Increase carbon flux |
| | synthetase | | from acetate to |
| | | | acetyl-CoA |
| E. coli | pgl: 6- | OE | Increase carbon flux |
| | phosphogluconolactonase | | to acetylphosphate |
| | (ybhE) | | (overexpression of |
| | | | the Entner- |
| | | | Doudoroff pathway) |
| E. coli | pkl: phosphoketolase | OE | Increase carbon flux |
| | | | to acetylphosphate |
| | | | (overexpression of |
| | | | the Entner- |
| | | | Doudoroff pathway) |
| E. coli | pgi: glucose-6-phosphate | OE | Increase carbon flux |
| | isomerase | | from G6P to F6P |
| E. coli | pta: phosphate | OE | Increase acetyl-CoA |
| | acetyltransferase | | from |
| | | | acetylphosphate |
| E. coli | ldhA: lactate | KO/KD | Reduce lactate |
| | dehydrogenase A | | production from |
| | | | pyruvate |
| E. coli | poxB: pyruvate oxidase | KO/KD | Reduce acetate |
| | | | production from |
| | | | pyruvate |
| E. coli | ackA: acetate kinase | KO/KD | Reduce acetate |
| | | | production from |
| | | | acetylphosphate |
| E. coli | adhE: alcohol | KO/KD | Reduce ethanol |
| | dehydrogenase enzyme | | production from |
| | | | acetyl-CoA |
| E. coli | did: D-lactate | KO/KD | Reduce lactate |
| | dehydrogenase | | production from |
| | | | pyruvate |
| E. coli | aceE: pyruvate | KD | Increase carbon flux |
| | dehydrogenase | | to MEP pathway |
| | | | from pyruvate |
| E. coli | ispA: geranyl | KD | Increase carbon flux |
| | diphosphate | | to DMAPP from IPP |
| | | | and reduce it to GPP |
| | | | from IPP |

With continued reference to FIGS. 3A-B, in one or more embodiments, promoter engineering may be used to improve the transcriptional level of a gene. Promoter engineering modifies the promoter region to increase gene expression levels, thereby improving enzyme production and activity. In one or more embodiments, codon optimization may be used to improve the translational efficiency of a gene. For the purposes of this disclosure, “codon optimization” is a technique used in genetic engineering to improve the expression of a gene in a particular host organism. Codon optimization involves altering the DNA sequence of a gene to use codons that are more frequently preferred by the host organism's translational machinery. A codon optimization process may take into account a codon bias of the host, ensuring that a synthetic gene sequence is translated more efficiently into the desired protein. Codon optimization may enhance the yield and function of a protein, which may be crucial for various applications in biotechnology and synthetic biology. It is worth noting that promoter engineering and codon optimization are distinct yet complementary techniques in genetic engineering. Promoter engineering involves modifying the promoter region of a gene to enhance its expression by improving the binding efficiency of transcriptional machinery. Codon optimization, on the other hand, focuses on altering the coding sequence of a gene to use preferred codons of the host organism, thereby improving translation efficiency. While both aim to increase protein production, promoter engineering targets transcriptional levels, and codon optimization targets translational efficiency. Combining both techniques may in some cases synergistically enhance an overall gene expression.

With continued reference to FIGS. 3A-B, in some cases, a gene encoding an enzyme may be silenced using Hfq-mediated RNA silencing. For the purposes of this disclosure, “Hfq-mediated RNA silencing” is a process wherein the Hfq protein facilitates a regulation of gene expression through RNA interactions, by binding to small regulatory RNAs (sRNAs) and messenger RNAs (mRNAs) to promote a formation of RNA duplexes. This interaction may enhance or inhibit a translation of target mRNAs, leading to gene silencing. In some cases, this process may be useful for post-transcriptional regulation and may be harnessed for genetic engineering, therapeutic interventions, and synthetic biology applications. As nonlimiting examples, to facilitate accumulation of isoprene, Hfq-mediated RNA silencing may be used against geranyl diphosphate synthase (IspA), farnesyl pyrophosphate synthetase (Erg 20), geranyl diphosphate synthase (GPPS), geranylgeranyl diphosphate synthase (BTS1), farnesyl diphosphate synthase (FPPS), malate synthase (encoded by AceB or MLS1), ATP-citrate lyase (CitE) including AclY, AclB, and AclA, and/or pyruvate dehydrogenase (encoded by PDH) including pyruvate dehydrogenase E1 component (encoded by AceE), among others, to downregulate its activity, consistent with details described above.

With continued reference to FIGS. 3A-B, in some cases, one or more pathways within schemes of embodiments 300a or 300b may be condensed, inhibited, rearranged, or otherwise modified. As a nonlimiting example, one or more genes encoding one or more enzymes may be modified to provide a phosphoketolase workaround, such as by upregulating glycolysis, the pentose phosphate pathway, or the Entner-Doudoroff pathway. As another nonlimiting example, one or more enzymes may be co-localized to create multiple reaction centers in tandem, thereby bypassing one or more competing side reactions, consistent with details described above. As another nonlimiting example, one or more genes encoding one or more enzymes may be modified to inhibit the phosphotransacetylase-acetate kinase (Pta-AckA) pathway to facilitate acetate reuptake in a Crabtree-positive enzyme. The Pta-AckA pathway is a key metabolic route in many bacteria and may be particularly significant under anaerobic conditions or when there is an excess of carbon sources, as it allows a cell to dispose of excess A-CoA and generate additional ATP. Inhibition of the Pta-AckA pathway may facilitate an acetate reuptake and improve the overall carbon efficiency of isoprene production. The Pta-AckA pathway involves two main enzymes: Pta catalyzes the conversion of A-CoA to acetyl phosphate, whereas AckA catalyzes the conversion of acetyl phosphate to acetate to generate ATP. By inhibiting these enzymes, the production of acetate may be reduced. Such reduction may be beneficial in preventing acetate accumulation that can be detrimental to cellular growth and productivity. Genetically engineered microbe may be configured to produce a Pta-AckA inhibitor. For the purposes of this disclosure, a “phosphotransacetylase-acetate kinase inhibitor” is a chemical compound that interferes with the activity of the phosphotransacetylase (Pta) and/or acetate kinase (AckA). Nonlimiting examples of Pta-AckA inhibitors may include deacetylase inhibitors such as tricostatin A and nicotinamide. Specifically, such inhibitors may function by regulating acetyl phosphate-dependent protein acetylation. As another nonlimiting example, the MEP and the MVA pathways may be balanced with each other. Specifically, leveraging the MEP and MVA pathways may enhance the yield and production efficiency of isoprene by optimizing the flux of metabolites, providing metabolic redundancy, and improving the robustness of the production strain under varying conditions. In some cases, such balanced pathways may be implemented using a shunt. Such balanced pathways may offer several benefits. As a nonlimiting example, such balanced pathways may lead to a more efficient utilization of substrates, thereby reducing waste and improving the carbon economy of a genetically engineered microbe. Additionally, as a further nonlimiting example, such balanced pathways may allow for finer control over metabolic fluxes through genetic and regulatory modifications, facilitating the tuning of pathway activities to match specific production needs. Overall, such balanced pathways may enhance the efficiency, robustness, and flexibility of isoprene production, making it more economically viable and sustainable for industrial applications. As another nonlimiting example, the MEP and the MVA pathways may be balanced by increasing a flux through the MEP pathway to reduce carbon loss to the TCA cycle; this strategy may lead to an increase in isoprene production. A theoretical calculation of isoprene yield based upon flux ratios through the MEP and MVA pathways suggests that isoprene yield may increase with an increasing flux along the MEP pathway, with a maximum yield of approximately 0.31 g of isoprene per gram of glucose when the flux ratio is approximately 60:40.

Referring now to FIG. 3C, FIG. 3C is an exemplary embodiment 300c of experimental results collected using genetically engineered E. Coli with downregulated ackA-pta, poxB, and IdhA genes. Sample TEB00 is prepared using a wild-type K12 E. coli strain, and the error bars are standard deviations calculated based on five independent measurements. Sample TEB14 is prepared using a genetically modified E. coli strain, with ldhA, ackA-pta, and poxB genes knocked out, and the error bars are standard deviations calculated based on eight independent measurements. Compared to TEB00, the concentration of lactate, acetate, and ethanol byproducts are reduced in TEB14 by a factor of between 1.5 and 5. This reduction indicates that downregulation of ldhA, ackA-pta, and poxB genes in E. coli effectively suppresses competing reactions that prevent the accumulation of isoprene and potentially improves the carbon efficiency of isoprene biosynthesis.

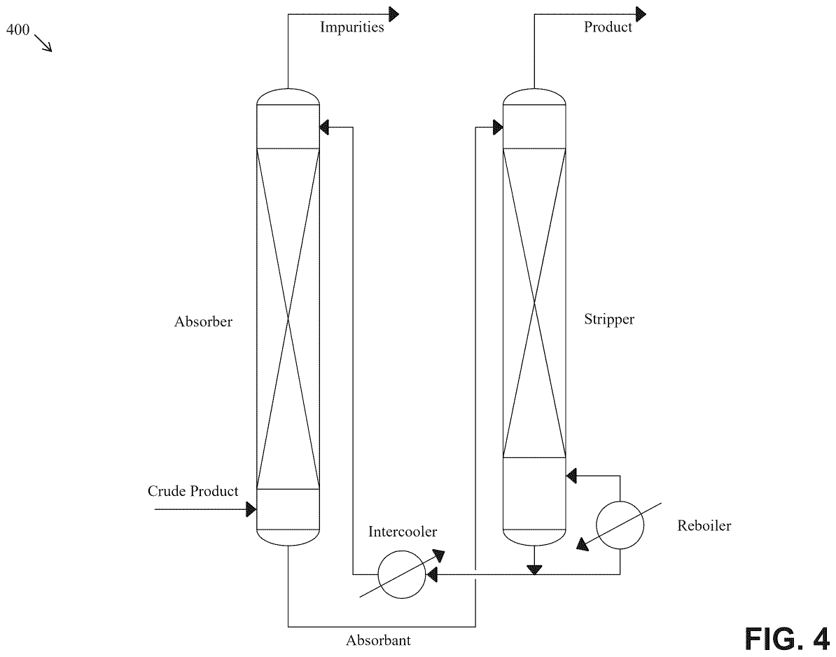

Referring now to FIG. 4, an exemplary embodiment 400 of an absorbing and stripping apparatus is illustrated. An absorbing and stripping apparatus includes a pair of absorbing and stripping columns. An absorbing and stripping column may operate by introducing a gas stream containing a target component at one end and an absorber, such as without limitation a liquid solvent, at the opposite end. An absorbing and stripping column is typically packed with structured packing or trays to enhance contact between gas and liquid phases, thereby increasing the efficiency of mass transfer. As gas ascends and liquid descends through an absorbing and stripping column, target component is absorbed into absorber, for example, a liquid phase (absorption) or stripped from the absorber into gas phase (stripping), achieving a desired separation or purification of components.

With continued reference to FIG. 4, in some cases, an absorbing and stripping apparatus may contain activated carbon to perform certain adsorption/desorption functions. For the purposes of this disclosure, “activated carbon”, also known as activated charcoal, is a highly porous form of carbon-based material with a large surface area. Activated carbon is often produced by processing carbonaceous materials, such as coconut shells, wood, or coal, among others, at high temperatures and then activating it using steam or chemicals to create a porous structure. The high adsorption capacity of activated carbon makes it a versatile absorber for various environmental, industrial, and health-related applications. As nonlimiting examples, activated carbon may be used in water purification to remove contaminants and odors, in air filtration to capture pollutants, in medical applications to treat poisonings and overdoses, in food and beverage processing to deodorize and decolorize products, and in gold recovery to extract gold from ores, among others.

With continued reference to FIG. 4, The choice of how an absorbing and stripping apparatus is implemented may depend on factors such as the percent isoprene in the off-gas, and the levels of other gases such as CO₂ and water vapor, and/or the scale of the recovery operation. A person of ordinary skill in the art, upon reviewing the entirety of this disclosure, will be able to recognize how an absorbing and stripping apparatus may be implemented for the invention described herein. Additionally, and/or alternatively, in some cases, an absorbing and stripping process may be complemented or at least partially replaced by compression/condensation, membrane permeation, or the like, consistent with details described elsewhere in this disclosure.

### EXAMPLES

### Example 1: Genetic Engineering of Clostridium ljungdahlii (C. ljungdahlii) for Isoprene Production

### Purpose of the Study

This study pertains to recombinant anaerobic acetogenic bacterial cells (Clostridium ljungdahlii) expressing a series of genes for the mevalonate (MVA) pathway and methylerythritol phosphate (MEP) pathway, which are controlled by carbon-specific regulator and promoter sequences to produce isoprene as a final product.

### INTRODUCTION

Isoprene biosynthesis is conducted through the mevalonate (MVA) pathway and methylerythritol phosphate (MEP) pathway, particularly in natural bacteria species. Described herein is the invention of a new metabolically engineered Clostridium ljungdahlii strains, which produce isoprene through an anaerobic culture system. This strain development includes implanting heterologous key enzymes of MVA and MEP pathways cloned from other species. It also includes the modified regulator/promoter clusters, which regulate the implanted gene cassettes for utilizing particular carbon sources. These recombinant regulator/promoter clusters are designed to work under a specific carbon source. Therefore, the recombinant regulator/promoter-harboring MVA and MEP key enzyme-coding gene cassette enables the biosynthesis of isoprene under relevant carbon sources in culture media through anaerobic culture methods.

### Summary of Study

The main modification in this study includes three sub-categories: i) implantation of the heterologous genes from other species for the critical enzymes in MEP and MVA pathways, ii) gene optimization for Clostridium ljungdahlii, and iii) recombinant regulator/promoter sequence for specific carbon utilization.

### Strategies

Implantation of the heterologous genes for key enzymes in the MVA and MEP pathways:

Beck et al. reported that a series of heterologous key enzymes in the MVA pathway had been implanted in the C. ljungdahlii, followed by successful isoprene biosynthesis through an anaerobic culture. Implanted key enzymes include diphosphomevalonate decarboxylase (MVD), mevalonate kinase (MK), phosphomevalonate kinase (PMK), mevalonate synthase (MvaS) from E. faecalis, acetoacetyl-coenzyme A thiolase/3-hydroxy-3-methylglutaryl-coenzyme A reductase (MvaE) from E. faecalis, isopentenyl diphosphate isomerase (idi) from S. cerevisiae, and isoprene synthase (IspS) from P. alba. These genes were codon-optimized for Clostridium species and sub-cloned in pertinent expression plasmid vectors. Similarly, Whited et al. reported that the heterologous protein in the non-mevalonate (MEP) pathway produces isoprene in E. coli. Therefore, a series of proteins in the MEP pathway of Clostridium ljungdahlii will be implanted. The enzymes for the MEP pathway include 1-deoxy-D-xylulose 5-phosphate synthase (DXS), 1-deoxy-D-xylulose 5-phosphate reductase (DXR), (E)-4-Hydroxy-3-methyl-but-2-enyl pyrophosphate synthase (IspG), (E)-4-Hydroxy-3-methyl-but-2-enyl pyrophosphate reductase (IspH, Anabaena), flavodoxin I (FldA), and ferredoxin NADP+ reductase (Fpr).

The C. ljungdahlii harbors the MVA pathway, MEP pathway, or MVA/MEP pathways for the isoprene biosynthesis. The regulator/promoter engineering strategy is a suitable tool for utilizing a particular carbon source from culture media. Biobutanol synthesis using a recombinant, specific regulator and promoter sequence with the essential enzymes for the butanol pathway has been reported.

Exemplary embodiments of specific genetic sequences pertaining to this study may include without limitation MvaE-C(SEQ ID No: 1), MvaS-C(SEQ ID No: 2), MK-C(SEQ ID No: 3), MVD-C(SEQ ID No: 4), PMK-C(SEQ ID No: 5), DXS-C(SEQ ID No: 6), DXR-C (SEQ ID No: 7), flda-C(SEQ ID No: 8), IspG-C-E. coli (SEQ ID No: 9), IspG-C-T. elong (SEQ ID No: 10), IspH-C(SEQ ID No: 11), petF-C(SEQ ID No: 12), petH-C(SEQ ID No: 13), idi-C (SEQ ID No: 14), IspS-C(SEQ ID No: 15) XyIR CAC3673 (SEQ ID No: 16), and xynB CAC3451 (SEQ ID No: 17), among others.

In this study, individual heterologous genes may be selected from various host species and cloned after codon optimization to the Clostridium species, which is the host strain of isoprene expression. Multiple genes may be cloned together to form cassettes in plasmid expression vectors. Recombinant regulator and promoter sequences may be searched and selected for particular carbon sources, such as without limitation xylose, arabinose, maltose, glucose, and fructose, among others. Then selected regulator/promoter sequences may be cloned in cloning vectors and combined with gene cassettes for the isoprene pathway in the same plasmid vectors. The regulator/promoter and isoprene-expressing cassette may be continuously located in the same vector. For example, a selected regulator and promoter set of XylR and xynB for xylose may be sub-cloned and combined with the MVA or MEP pathways gene cassettes. In detail, XyIR (CAC3673, SEQ ID NO: 16) derived from Clostridium acetobutylicum may be cloned for the regulator region, and xynB (CAC3451, SEQ ID NO: 17) derived from Clostridium celluloltyicum may be cloned for the promoter region.

The reporter protein expression system is tested to confirm the function of the regulator/promoter sequence for particular carbon sources in C. ljungdahlii. In other words, the beta galactosidase-expressing gene is located after the regulator/promoter sequence in the expression vector, followed by transformation into C. ljungdahlii. Following a general culture protocol, transformant C. ljungdahlii is cultured in proper nutrition culture media under anaerobic environments. The culture protocol includes a gas composition of 5% hydrogen, and 5% carbon dioxide, balanced with nitrogen gas at 30° C. Incubation may proceed for 1-3 days without agitating or shaking. A correct expression of the beta-galactosidase from the regulator/promoter under a proper carbon source may be optically confirmed by the blue color of the culture media after a successful expression of cultivation.

When the regulator/promoter functions correctly, the cloned regulator/promoter/cassette fragments may be sub-cloned to a pCJ24-1 plasmid vector. A pAM 120 plasmid vector may contain the Tn916 transposon components, so it is possible to introduce target DNA fragments to the genomic DNA of transformants, at a random position within the genomic DNA of the C. ljungdahlii. Successful transformants harboring the reg/pro/MVA-MEP/pCJ24-1 vectors may be treated under proper procedures to induce integration of target gene fragments. Screening for a successful gene integration to the genomic DNA of C. ljungdahlii may be performed through PCR amplification. Culture parameters for developed C. ljungdahlii strain, such as without limitation temperature, culture media compositions, and/or the concentration of carbon sources, etc., may be optimized at a flask level in an anaerobic chamber. Produced isoprene may be measured and analyzed using gas chromatography.

Optimized culture parameters may be applied to a stirred tank reactor of ≥0.5 L culture size. A similar or modified isoprene purification protocol may be applied to obtain isoprene from C. ljungdahlii that has been previous used for isoprene production by E. coli under anaerobic conditions.

Materials and methods

### Materials

C. ljungdahlii is purchased from the American Type Culture Collection (ATCC, USA). The ATCC Medium 2107 and Brucella agar to grow the C. ljungdahlii are purchased from VWR or other vendors.

The clostridial growth medium (CGM) consists of 2 g (NH₄) 2SO₄, 0.5 g KH₂PO₄, 1 g K₂HPO₄, 0.01 g MnSO₄: 2H₂O, 0.1 g MgSO₄·7H₂O, 0.015 g FeSO₄·7H₂O, 0.01 g CaCl₂), 0.02 g CoCl₂, 0.02 g ZnSO₄, 2 g tryptone, 1 g yeast extract, and 20 g glucose per liter in an anaerobic chamber. The CGM media may be used to analyze overall culture processes of (′. ljungdahlii after an initial revival step. Escherichia coli (E. Coli) is purchased from NEB. It is grown in a Luria-Bertani (LB) medium that contains 10 g tryptone, 10 g sodium chloride, and 5 g yeast extract per 1 liter of deionized water as the final volume.

### Methods

### Strain Preparation

C. ljungdahlii is purchased from the ATCC. Delivered strain is revived following the ATCC's handling procedure. A vial containing a C. ljungdahlii pellet is opened in an anaerobic chamber. 0.5 mL of #2107 broth is added to resuspend the pellet. The resuspended cell is transferred to a 15-mL centrifuge tube that contains 5 mL of #2107 media. Before cell revival, the #2107 media stays in the anaerobic chamber for gas exchange. Several drops of resuspended cell are spread on the Brucella blood agar plate. And the plates are incubated in a 37° C. chamber for 48 or 72 hours until a proper size of colonies becomes visible. One agar plate is incubated in aerobic circumstances to confirm the contamination in a 37° C. chamber. Successful culture samples are aliquot and mixed with 30% glycerol as a final glycerol cell stock preparation concentration and stored at −80° C. in a freezer.

### Gene Cloning

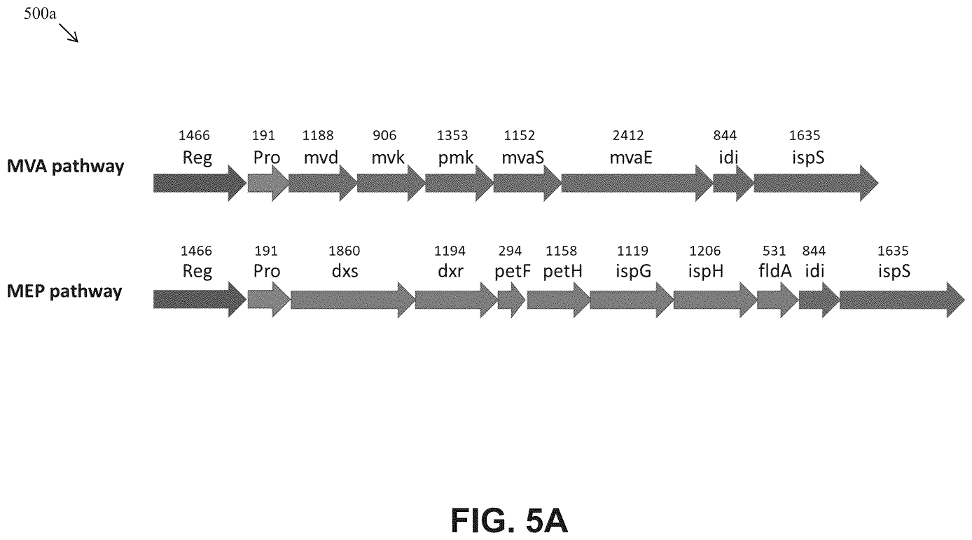

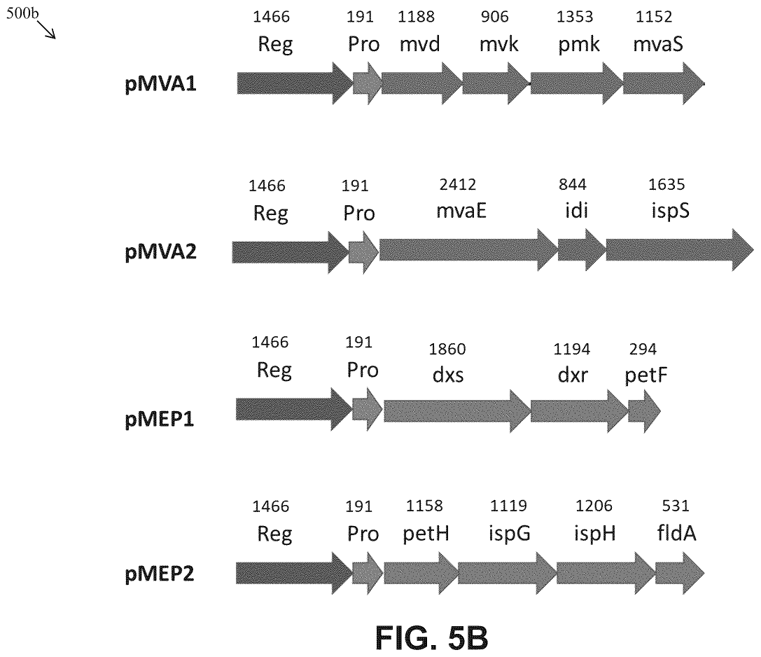

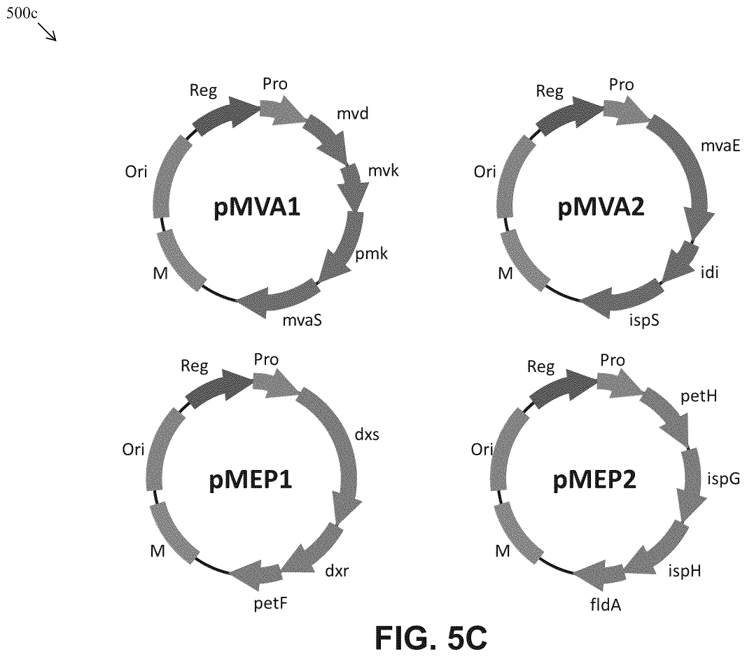

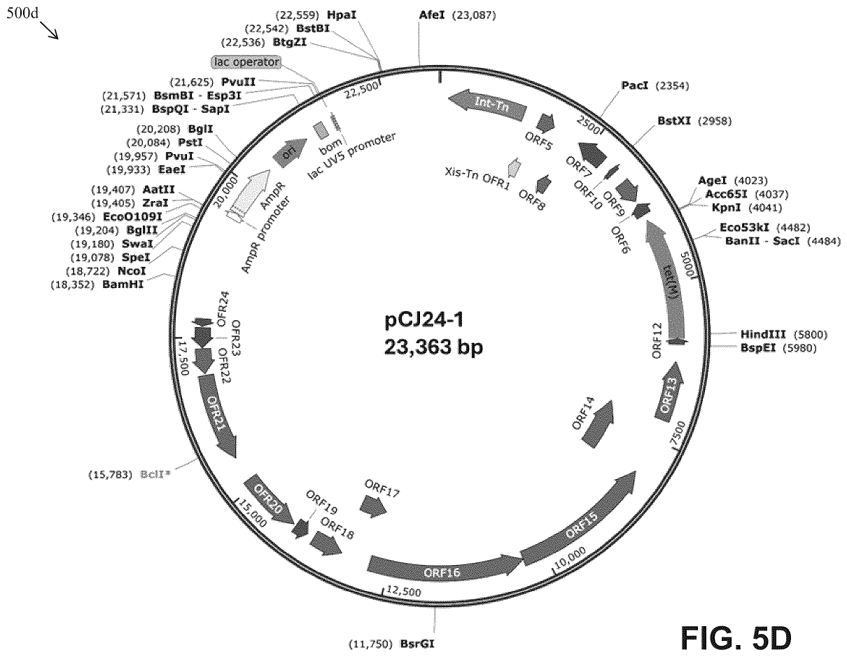

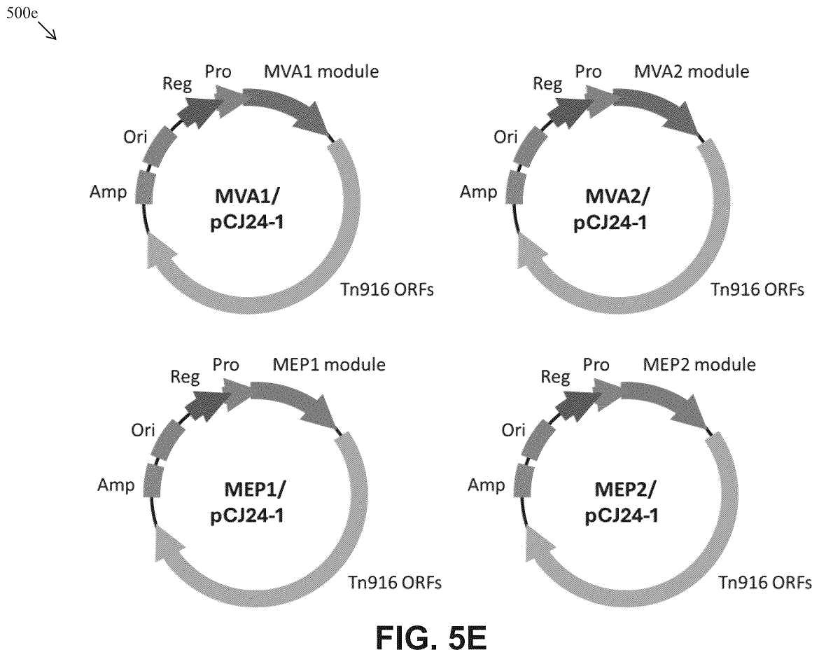

Recombinant enzymes in the MVA and MEP pathways are codon optimized for Clostridium species using software and artificially synthesized in plasmid backbone vectors by vendors. The enzyme coding genes for the MVA pathway include without limitation MvaS, MvaE, idi, IspS, MK, PMK, and MVD. Their estimated nucleotide lengths are 1152 bp for MvaS (SEQ ID NO: 2), 2412 bp for MvaE (SEQ ID No: 1), 1356 bp for PMK (SEQ ID No: 5), 549 bp for idi (SEQ ID NO: 14), 1635 bp for IspS (SEQ ID NO: 15). Similarly, enzyme coding genes for the MEP pathways include DXS, DXR, IspG, IspH, fldA, and fpr, but are not limited thereto. Their estimated nucleotide lengths are 1863 bp for DXS (SEQ ID NO: 6), 1197 bp for DXR (SEQ ID NO: 7), 852 bp for IspE, 480 bp for IspF, 1119 or 1209 bp for IspG (SEQ ID NO: 9-10), 1209 bp for IspH (SEQ ID NO: 11), and 531 bp for fldA (SEQ ID NO: 8). The individually synthesized genes in plasmid backbone samples may be revived via gene transformation into the K12 E. coli competent cells (see FIG. 5A. embodiment 500a).

To conduct the transformation, the K12 E. coli competent cells are prepared for the heat-shock transformation method using a typical 100 mM ice-cold calcium chloride solution. Then, approximately 5 ng of artificially synthesized plasmid samples is added to 90 μL of competent cells in microcentrifuge tubes, followed by gentle tapping for mixing plasmid DNA with cells. Mixed cells are incubated in a 42° C. water bath for 90 seconds, quickly transferred in ice, and allowed to sit for 120 seconds. Afterwards, 1 mL of warm SOC nutrient liquid media is added to the cells and mixed by gently inverting of the tubes, followed by incubation at 37° C. and shaking incubator for 90 minutes. The well-grown cells are spread on LB agar plates harboring proper antibiotics against plasmid vectors, then will be incubated in a 37° C. chamber overnight. Successful colonies from the plates will be inoculated in LB nutrient media with proper antibiotics and fully grown in a 37° C. shaking incubator overnight.

Regulator and Promoter Cloning with Reporter Gene

A selected regulator and promoter set for xylose is sub-cloned with the MVA or MEP pathways gene cassettes. XyIR (CAC3673, SEQ ID NO: 16), derived from Clostridium acetobutylicum, is the regulator region, and xynB (CAC3451, SEQ ID NO: 17), derived from Clostridium celluloltyicum, is the promoter region. The estimated size of the XylR regulator fragment is 1466 bp (SEQ ID NO: 16), and the xynB promoter is 191 bp (SEQ ID NO: 17). These two fragments may be artificially synthesized or may be cloned from Clostridium species through PCR amplifications. The obtained fragments are subcloned in commercial cloning vectors. Additional nonlimiting examples of promotors, regulatory fragments, and effectors of expression associated thereto are provided in Table 2 below.

TABLE 2
Promotors, Regulatory Protein Genes, and Effectors
of Expression Associated Thereto.

| Promoters | Regulatory Protein | Small-Molecule Effector |
|---|---|---|
| CAC2610-12 | XylR (CAC3673) | Xylose |
| CAC3451-2 | | |
| Ccell133 | | |
| CAC0231, 0234 | FruR (CAC0231) | Fructose |
| CAC1341-2 | AraR (CAC1340) | Arabinose |
| CAC1343 | | |
| CAC0423-5 | LicT (CAC0422) | Sucrose |
| CAC0532-3 | CcpA (CAC0531) V302N | Maltose |
| | mutant | |

The successfully cloned regulator-promoter fragment is subcloned with a reporter gene, such as the green fluorescent protein (GFP)-encoding gene. The GFP-encoding gene may be codon-optimized to Clostridium species for the best expression patterns. The reporter gene will be expressed under proper carbon sources related to the regulator/promoter set. For example, the XylR-xynB regulator/promoter set produces the GFP gene expression when the xylose is present in the culture media.

### Sub-Cloning

The successful cloned regulator/promoter sequence is combined with the synthesized genes described above. The proper bacterial cloning plasmid vector is used as the backbone plasmid for the sub-cloning processes. Considering the cloning efficiency, the size of an inserted gene fragments is around 7 kb, and the total size of the plasmid vectors will be around 10-12 kb (see FIG. 5B, embodiment 500b). Four plasmids are cloned, pMVA1, pMVA2, pMEP1, and pMEP2 that include modules of regulator-promoter-MVD-MK-PMK-MvaS, regulator-promoter-MvaE-idi-IspS, regulator-promoter-DXS-DXR-petF, and regulator-promoter-petH-IspG-IspH-fldA, respectively (see FIG. 5C, embodiment 500c). The cloning procedure is performed using a restriction enzyme cloning method or the Gibson Assembly cloning method. The successful vector constructs are transformed into proper competent cells, such as K12 E. coli, or the like, and selected on LB/agar plates screening. Plasmid vectors are purified from the selected colonies, and the sequence are confirmed through Sanger sequencing analysis and restriction enzyme digestion pattern analysis. Constructed modules are cloned in a pCJ24-1 plasmid vector (see FIG. 5D, embodiment 500d) containing a Tn916 transposon gene cluster for future genomic DNA integration to C. ljungdahlii. The tetracycline resistance marker (TetR) gene in the pCJ24-1 vector is replaced with the reg-pro-module cassettes. The ampicillin resistance marker (AmpR) gene remains in the pCJ24-1 vector. Each module is individually integrated into the pCJ24-1 plasmid, followed by a construction of MVA1/pCJ24-1, MVA2/pCJ24-1, MEP1/pCJ24-1, and MEP2/pCJ24-1. Each vector construct size is approximately 27-30 kb. Since the size of pCJ24-1-based vectors is relatively large, a proper competent cell, such as K12 E. coli, may be considered a candidate for competent cells for the cloning process (see FIG. 5E, embodiment 500e).

Transformation to C. ljungdahlii

The C. ljungdahlii cell is cultured in the anaerobic chamber using the CGM media. For the electrocompetent cell preparation, 1 mL of fully cultured C. ljungdahlii cell is resuspended in an ice-cold ETM buffer that includes 270 mM sucrose, 0.6 mM Na₂HPO₄·12H₂O, 4.4 mM NaH₂PO₄·2H₂O, 10 mM MgCl₂·6H₂O. Then, the cell is resuspended in 1 mL of ET buffer that includes 270 mM sucrose, 0.6 mM Na₂HPO₄·12H₂O, 4.4 mM NaH₂PO₄·2H₂O. The methylated four pCJ24-1 plasmid constructs (MVA1-, MVA2-, MEP1-, and MEP2-pCJ24-1) are transformed in C. ljungdahlii under 2000 V and 25 μF capacitance using a 4-millimeter cuvette. Transformed cells are mixed with a pre-warmed 2× YTG media comprising 10 g yeast extract, 16 g tryptone, 5 g glucose, 4 g NaCl per liter, at pH 5.8, followed by overnight incubation in the anaerobic chamber. The incubated cells are spread on the 2× YTG agar plates and incubated at 37° C. in the anaerobic chamber. Gene insertion is confirmed through PCR analysis using randomly selected colonies from agar plates.

Isoprene Production from the Developed Strains

Isoprene synthesis is conducted using confirmed C. ljungdahlii transformants from the previous step. Transformants are grown in the CGM media containing xylose as a carbon source at a flask level. The concentration of xylose is tested within a wide range to obtain the best productivity from the culture. When the best cultivation parameters are obtained from the flask level, the selected samples are cultured in scale-up size fermenters, such as 3-L, 10-L, 100-L, and larger sizes with the optimized culture parameters. In the fermenters, anaerobic gas circumstances are regulated using an external gas influx at a proper rate.

### Example 2: Increase Isoprene Titer Using Via Downregulation of IspA and AceE Genes in Escherichia coli Genomic DNA

### Background

Competing metabolic pathways may affect the flux from a carbon source to target products. In the case of isoprene biosynthesis, two of the competing pathways are catalyzed by geranyl diphosphate/farnesyl diphosphate synthase (IspA, 299 amino acids) and pyruvate dehydrogenase (AceE, 887 amino acids), respectively. IspA converts isopentenyl diphosphate (IPP) and dimethylallyl diphosphate (DMAPP) to geranyl diphosphate (GPP) and farnesyl diphosphate (FPP), consistent with details described above. AceE converts pyruvate to acetyl Co-A, which may be subsequently consumed in the TCA cycle. As a result, both IspA and AceE may result in a lower carbon efficiency in isoprene synthesis.

Downregulation of ispA and AceE may increase the carbon flux from glucose to isoprene. Since a complete removal of these specific genes by knocking them out from the genomic DNA may raise the risk of cell lethality, it may be preferred to apply incomplete inhibition using a knockdown method that removes mRNA but maintains low expression levels of a target enzyme. Specifically, downregulation of an IspA enzyme-encoding gene may increase the concentration of DMAPP, thus allowing for an increased yield of isoprene. Similarly, downregulation of AceE may likely reduce the loss of carbon through the TCA cycle. However, reducing AceE activity may increase the risk of depressing the MVA pathway as well due to a reduction in acetyl-CoA concentrations. Nevertheless, increasing flux through the MEP pathway with a reduced carbon loss to the TCA cycle may lead to an increase in isoprene production. A theoretical calculation of isoprene yield based upon flux ratios through the MEP and MVA pathways suggests that isoprene yield may increase with an increasing flux along the MEP pathway, with a maximum yield of approximately 0.31 g of isoprene per gram of glucose when the flux ratio is approximately 60:40.

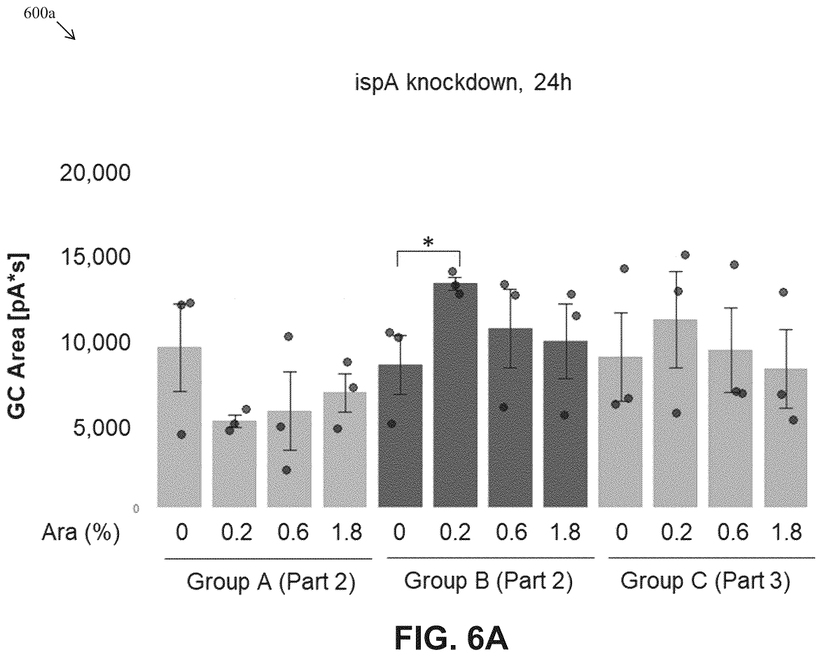

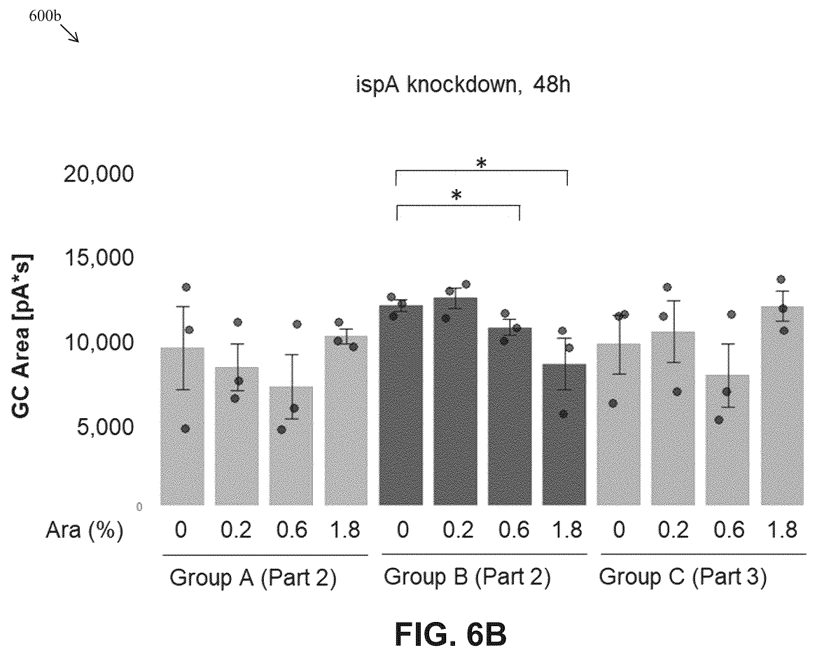

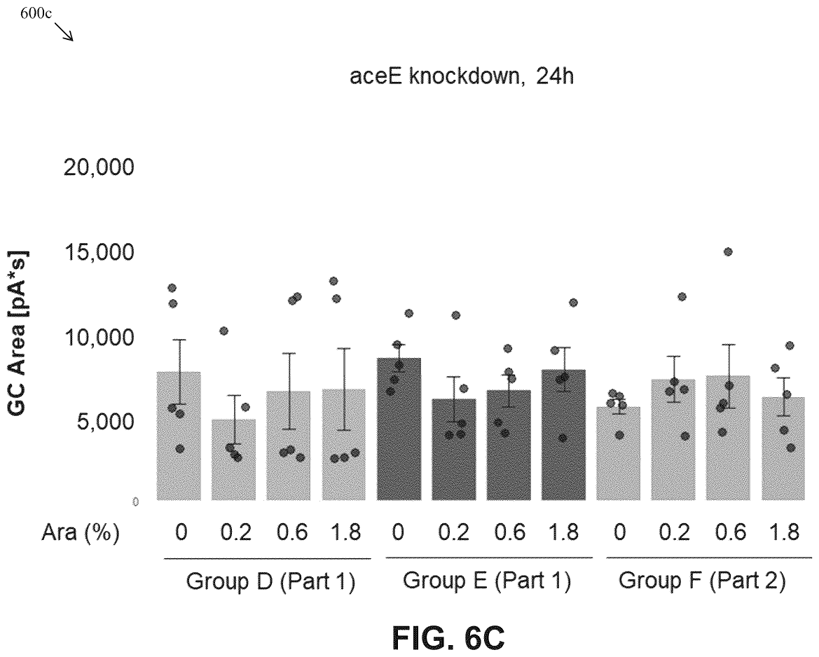

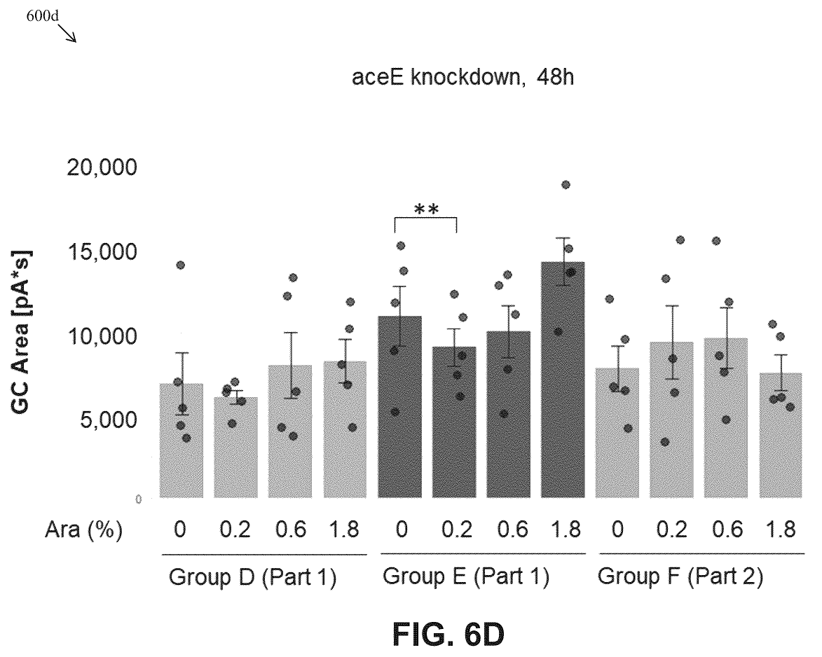

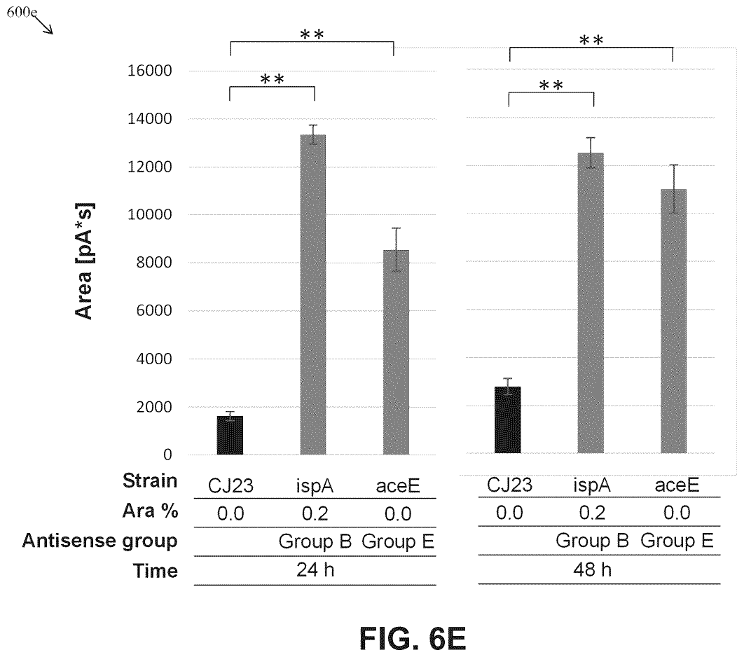

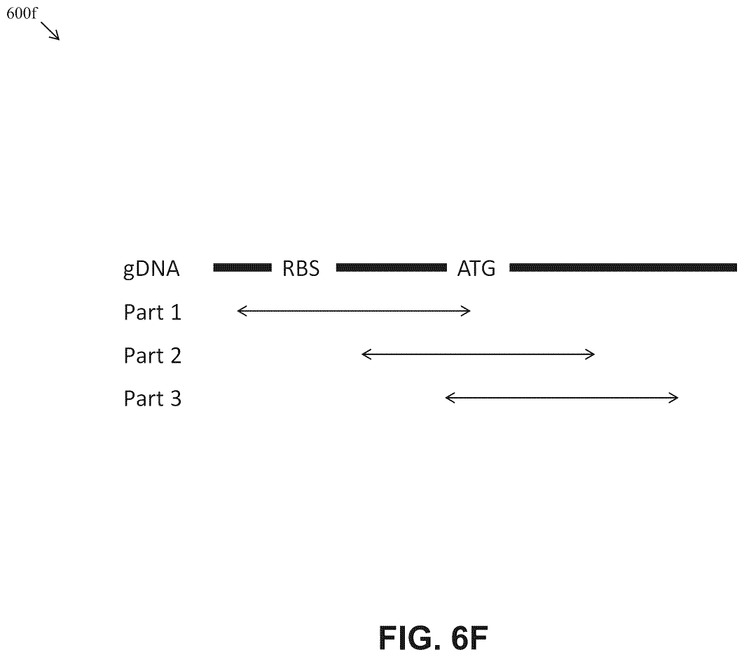

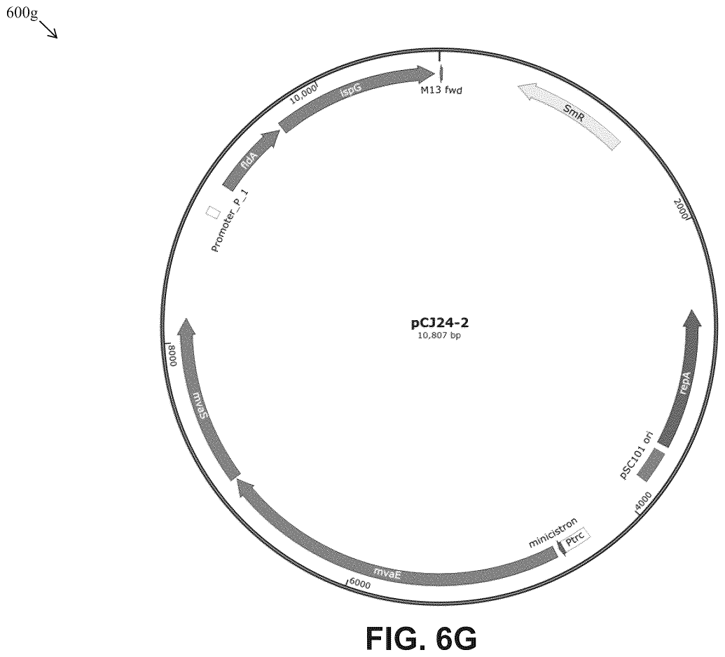

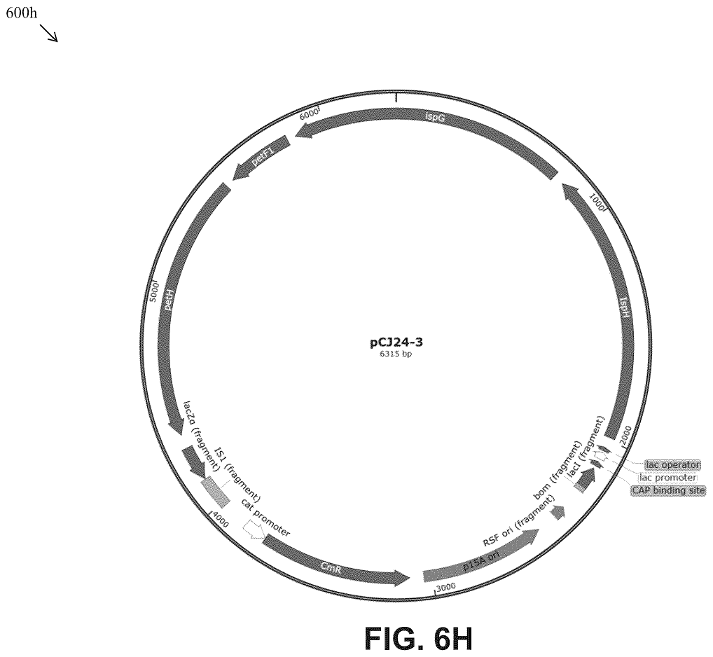

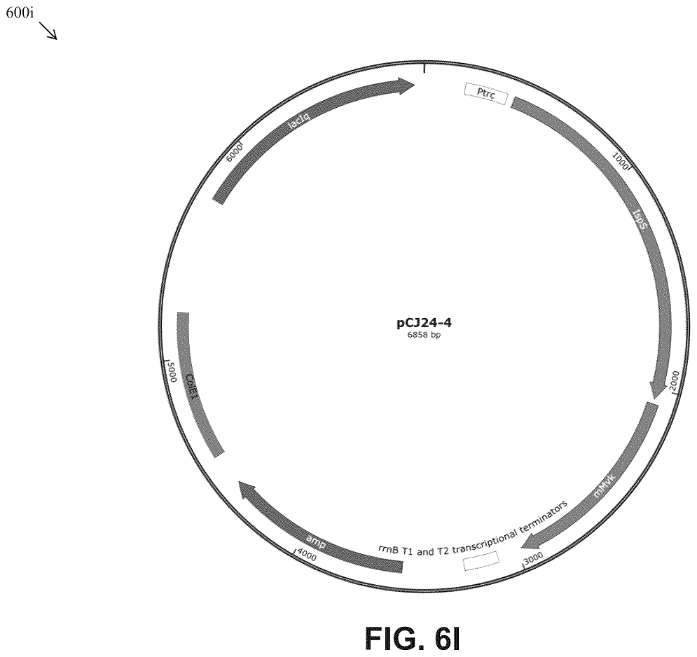

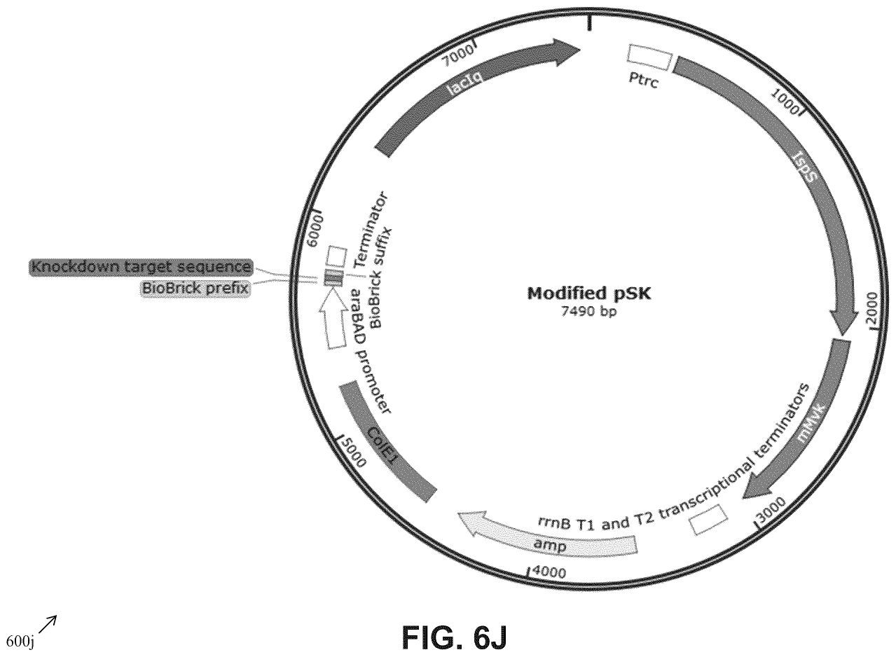

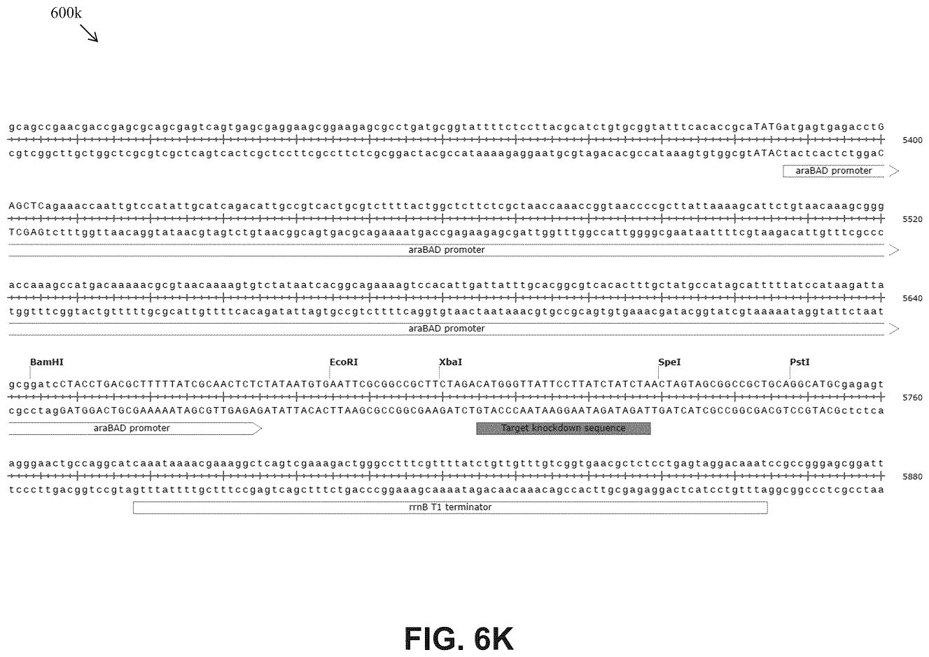

CJ23 is used as a reference strain and serves as a benchmark for evaluating the efficiency of isoprene biosynthesis. As described in the materials and methods below, CJ23 contains three plasmids encoding genes required for efficient isoprene production (see FIGS. 6G-J). One of the plasmids, pCJ24-4, may be modified to express an antisense RNA (asRNA) under an arabinose-inducible promoter to target and/or downregulate the activity of either IspA or AceE.

### IspA Knock-Down

Three groups of IspA knockdown strains are selected from pre-screening tests involving multiple single-colony inoculations and fermentations using a traditional protocol with Hungate tubes. Among the three asRNA target regions within the IspA-encoding gene, knockdown of the region centered on the ATG (Groups A and B) and the region downstream from the ATG (Group C) efficiently increases isoprene titers. In contrast, the asRNA targeting the RBS region does not contribute to an increase in isoprene titers. These three selected strains are then tested again in triplicate. The isoprene titer is measured using gas-chromatography (GC) at 24 and 48 hours, and the collected data are analyzed, see FIGS. 6A-B, embodiments 600a-b.

After 24 hours of fermentation, Groups B and C show that a 0.2% arabinose induction generates higher titers than other concentrations, with only Group B showing a statistically significant difference between a 0% and a 0.2% induction. However, Group A exhibits the opposite pattern compared to Groups B and C. Typically, arabinose induction concentrations range from 0.02% to 1%. The data from Group A show that samples with no-induction (0%) and over-dose induction (1.8%) have higher isoprene titers compared to commonly used 0.2% and 0.6% concentrations. After 48 hours of fermentation, the levels of isoprene production are elevated and show no dependence on the amount of arabinose added. These results suggest that the asRNA is being expressed in the absence of arabinose, likely due to a ‘leaky’ promoter.

### AceE Knock-Down

The asRNA fragments target three parts of the AceE-encoding gene, consistent with details described above pertaining to IspA. Knocking down the region targeting the RBS region (Groups D and E) and the region centered on the ATG (Group F) appears to increase isoprene production (see Figure FIGS. 6C-D, embodiments 600c-d), while knockdown of the region downstream from the ATG showed less efficiency than other groups. In some cases, the titer of isoprene is considerably higher than what is observed with the CJ23 reference, but the isoprene production levels show no difference in response to arabinose induction (see Figure FIGS. 6C-D, embodiments 600c-d). These results suggest that the asRNA is being expressed in the absence of arabinose, perhaps due to a ‘leaky’ promoter.

### Conclusion

In nearly all cases, the isoprene titers are considerably higher than what is typically observed with CJ23 reference. Specifically, the IspA Group B strain under 0.2% induction and AceE Group E strain under 0% induction demonstrate significantly higher titers compared to CJ23 (see FIG. 6E, embodiment 600e).

### Materials and Methods

### Knock-down Strategy

The 17 bp antisense RNA (asRNA) fragments are designed to target three areas at the 5′ region of the open reading frames (ORFs). Based on the position of the ATG start codon in the ORF, it is divided into three parts: a region including the ribosome binding site (RBS) upstream of the ATG (Part 1), a region centered on the ATG (Part 2), and a region downstream starting from the ATG (Part 3) (see FIG. 6F, embodiment 600f). The synthesized asRNA fragments and an araBAD promoter controlled by arabinose are cloned into the pCJ24-4 vector. The successfully cloned pCJ24-4 asRNA vectors containing the synthesized asRNA fragments are confirmed through sequencing analysis and introduced into CJ23 host cells by the heat-shock method, generating modified CJ23 strains. FIGS. 6G-J, embodiments 600g-j show vector maps for the pCJ24-2, pCJ24-3, and pCJ24-4 vectors for the CJ23 and the modified pCJ24-4 vector for the knockdown tests. FIG. 6K, embodiment 600k includes an exemplary sequence composition (SEQ ID NO: 18) for the knockdown, with the araBAD promoter, the rrnB T1 terminator, target knockdown sequence region highlighted.

### Small Scale Fermentations

Following a standard fermentation protocol, these modified CJ23 strains are fermented to produce isoprene in Hungate tubes. In brief, 10 successfully grown colonies are selected from LB agar plates containing appropriate antibiotics and inoculated in 5 mL of LB media with antibiotics. This step is followed by overnight culture at 37° C. in a 250-rpm shaking incubator. The fully grown cells are subcultured in 5 mL LB under the same conditions until the optical density at 600 nanometers (OD600) reaches the target levels, such as 0.4, 0.8, and 1.2. The asRNA expressions are controlled by an arabinose-inducing system. At proper target ODs, 640 μL of freshly grown cells are transferred into Hungate tubes, with 10 g/L glucose, 1 mM IPTG, and 0, 0.2, 0.6, and 1.8 wt % concentrations of arabinose. A butyl stopper cap and a screw cap are firmly sealed on the Hungate tubes, which are then cultured at 30° C. in a shaking incubator. At 24 h and 48 h post-induction, 200 cm³ of gas is collected from the headspace of the Hungate tube using a 250 mL GC syringe (Agilent) and analyzed using GC (7890B, Agilent).

### Example 3: Large-Scale Fermentation Study of Isoprene Using the MVA Pathway of a Genetically Engineered E. Coli

### Background

Isoprene may be produced natively in the chloroplasts of plants through the MEP pathway. However, isoprene precursors may also be synthesized in cells through the MVA pathway, and a microbe may be genetically engineered to produce isoprene through both pathways.

### Results

Multiple transformations have been performed to introduce plasmids into E. coli K12 E. coli cells. These plasmids contain genes encoding enzymes along the MVA pathway.

Specifically, plasmids containing genes encoding enzymes along the MVA and/or MEP pathway were transformed into K12 E. coli chemically competent cells. Colonies were then selected and grown in liquid culture (2 mL LB+antibiotics+glucose) at 37° C. until the optical density (OD) reached approximately 0.6. At this point, 790 μL of cells were transferred to 20 mL headspace autosampler vials and 7.9 μL of 100 mM Isopropyl B-D-1-thiogalactopyranoside (IPTG) was added. The vials were crimp sealed, incubated at 30° C. for approximately 48 hours, subsequently placed in an autosampler tray, and analyzed using the headspace autosampler connected to a gas chromatography with flame ionization detection (GC-FID) instrument. Each group of genetically engineered E. Coli (i.e., with or without a complete MVA pathway) includes 20 separately prepared samples (i.e., biological replicates).

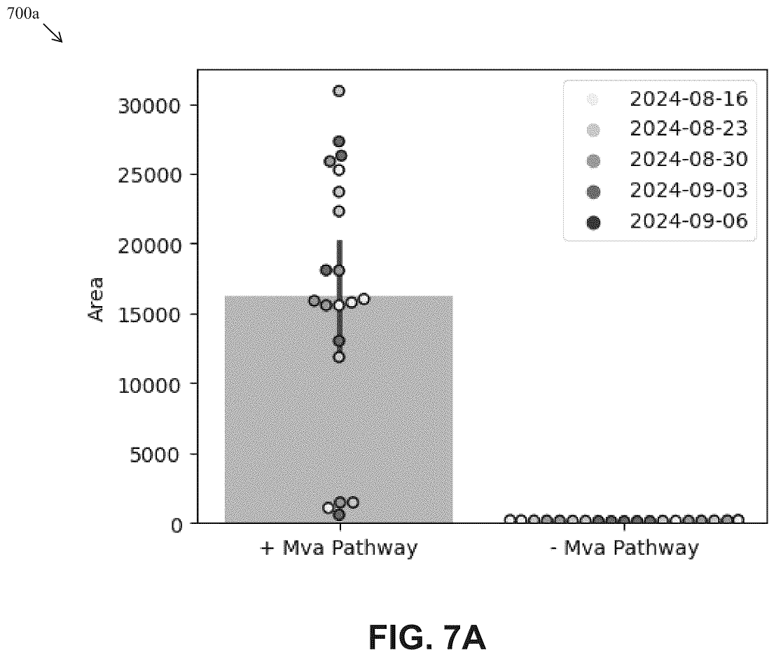

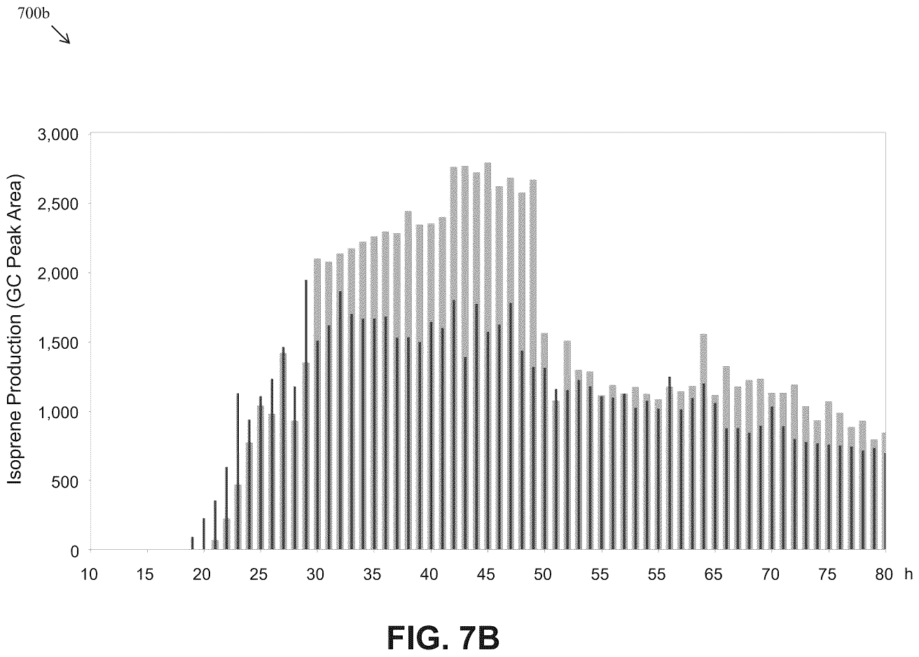

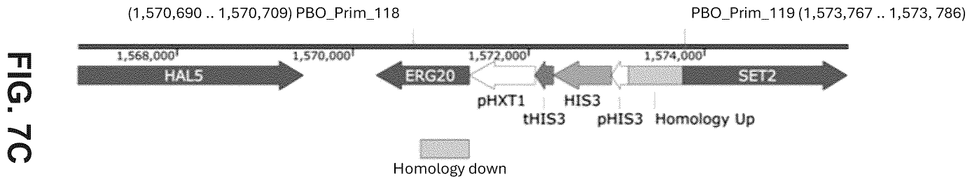

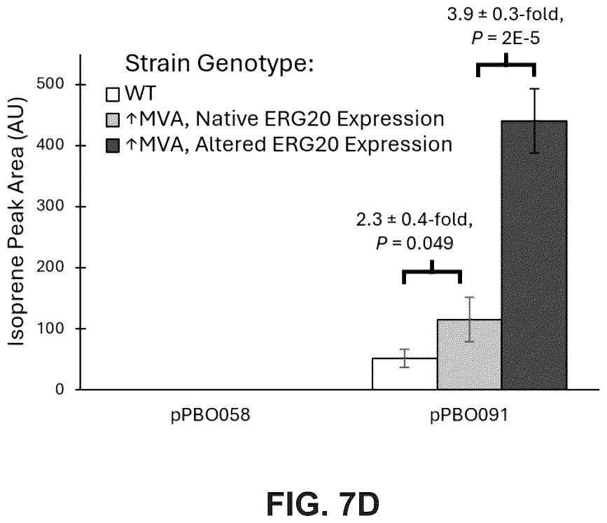

Referring now to FIG. 7A, FIG. 7A includes experimental results 700a showing isoprene production levels in genetically engineered E. Coli strains with and without enzymes along the MVA pathway; each point represents a unique biological sample (colony). Legends are based on the dates on which GC measurements were collected. When all enzymes along the MVA pathway (as well as an IspS enzyme) are included on the plasmids, there is an observable production of isoprene. In contrast, when only a subset of these enzymes is present, isoprene is not produced. Average peak areas for different samples are summarized in Table 3 below.

TABLE 3
Average Peak Areas for Different Samples with or
without the MVA Pathway, Measured Using GC-FID.

| Sample | Average Area | No. of Measurements |
|---|---|---|
| With Complete MVA | 16302 ± 9333 | 20 |
| Pathway | | |
| Without Complete MVA | 158 ± 21 | 20 |
| Pathway | | |

E. coli strains transformed with genetic sequences encoding enzymes along both the MEP and MVA pathways also produced isoprene. Such E. Coli strains were cultured by fermentation in stirred tank reactors at a larger scale.

Referring now to FIG. 7B, FIG. 7B includes experimental results 700b from two independent trials showing integrated gas chromatography (GC) peak areas measured after every hour of fermentation over the course of 80 hours.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A method of producing a dimethylcyclooctane-based aviation fuel from isoprene, the method comprising:
i) culturing a genetically engineered yeast under suitable conditions via one or more metabolic pathways to produce a first product comprising isoprene, wherein:
the genetically engineered yeast comprises a heterologous nucleic acid construct encoding a peptide, wherein the peptide comprises an activity configured to contribute to an enhanced production of isoprene, wherein the activity comprises at least a catalytic activity configured to be upregulated or downregulated;
ii) dimerizing isoprene, using a transition metal-based catalyst, to form a second product comprising dimethylcyclooctadiene; and
iii) hydrogenating dimethylcyclooctadiene, using a hydrogenation catalyst, to form a third product comprising dimethylcyclooctane.

2. The method of claim 1, further comprising, between step i) and ii):
iv) purifying the first product, using a first purification apparatus, to isolate isoprene.

3. The method of claim 2, wherein the first purification apparatus comprises an absorbing and stripping apparatus.

4. The method of claim 3, wherein the absorbing and stripping apparatus comprises activated carbon.

5. The method of claim 1, further comprising, between step ii) and iii):
v) purifying the second product, using a second purification apparatus, to isolate dimethylcyclooctadiene from at least a byproduct.

6. The method of claim 5, wherein the second purification apparatus comprises a distillation apparatus.

7. The method of claim 5, wherein the at least a byproduct comprises at least a Diels-Alder cycloaddition product.

8. The method of claim 1, wherein the genetically engineered yeast comprises bacteria or fungi.

9. The method of claim 1, wherein the genetically engineered yeast includes one or more members selected from a group consisting of Escherichia Coli, Vibrio natriegens, Saccharomyces cerevisiae, Clostridium ljungdahlii, and Aspergillus.

10. The method of claim 1, wherein the peptide includes one or more members selected from a group consisting of 1-deoxy-D-xylulose-5-phosphate synthase (DXS), isoprene synthase (IspS), isopentenyl diphosphate isomerase (idi), phosphatase, hydrolases including Nudix hydrolase, 3-hydroxy-3-methylglutaryl-coenzyme A reductase (HMGR), 3-methyl-3-buten-1-ol dehydroxylase (OhyA) and OhyA-like enzymes, (E)-4-hydroxy-3-methylbut-2-enyl diphosphate synthase (IspG), 1-deoxy-D-xylulose 5-phosphate reductase (DXR), (E)-4-hydroxy-3-methylbut-2-enyl diphosphate reductase (IspH), acetyl-CoA C-acetyltransferase/acetoacetyl-coenzyme A thiolase (MvaE), HMG-COA Synthase (MvaS), acylating acetaldehyde dehydrogenase (A-ALD), transcription factor (UPC2), mevalonate kinase (MK), aldehyde dehydrogenase (ALD5), acetyl-CoA synthetase including ACSA, ACS1, and ACS2, pantothenate kinase (CAB1), dihydrolipoamide acetyltransferase component of pyruvate dehydrogenase complex (LAT1), glucose-6-phosphate dehydrogenase (zwf), 6-phosphogluconolactonase (ybhE), phosphoketolase (pkl), glucose-6-phosphate isomerase (pgi), and phosphate acetyltransferase (pta), geranyl diphosphate synthase (IspA), geranyl diphosphate synthase (GPPS), farnesyl diphosphate synthase (FPPS), pyruvate dehydrogenase (PDH) including pyruvate dehydrogenase E1 component (AceE), pyruvate oxidase (poxB), pyruvate decarboxylase (PDC), malate synthase (AceB), citrate synthase (CIT1), ATP-citrate lyase (CitE) including AclY, AclB, and AclA, glycerol-producing enzymes including cGPDH and G3PP [41], ethanol-producing enzymes including invertase and zymase, alcohol dehydrogenase (ADH1), alcohol dehydrogenase enzyme (adhE), lactate-producing enzymes including lactate dehydrogenase A (IdhA) and D-lactate dehydrogenase (did), acetate-producing enzymes including acetyl coenzyme A (acetyl-CoA) acetate kinase (ackA), and an enzyme providing a phosphoketolase workaround.

11. The method of claim 1, wherein the dimethylcyclooctadiene comprises one or more members selected from a group consisting of 1,5-dimethyl-1,5-cyclooctadiene, 1,6-dimethyl-1,5-cyclooctadiene, 2,6-dimethyl-1,3-cyclooctadiene, 2,6-dimethyl-1,4-cyclooctadiene, 3,7-dimethyl-1,5-cyclooctadiene, 3,7-dimethyl-1,3-cyclooctadiene, and 3,6-dimethyl-1,3-cyclooctadiene.

12. The method of claim 1, wherein the dimethylcyclooctane comprises one or more members selected from a group consisting of cis-1,4-dimethylcyclooctane, trans-1,4-dimethylcyclooctane, cis-1,5-dimethylcyclooctane, and trans-1,5-dimethylcyclooctane.

13. The method of claim 1, further comprising:
vi) filtrating the third product, using a first filter, to form a first purified product.

14. The method of claim 13, wherein the first filter comprises a pore size between 100 nanometers and 10 micrometers.

15. The method of claim 14, wherein the first filter comprises a pore size of 1 micrometer.

16. The method of claim 13, further comprising:
vii) filtrating the first purified product, using a second filter, to form a second purified product.

17. The method of claim 16, wherein the second filter is a clay filter.

18. The method of claim 1, wherein the transition metal-based catalyst includes one or more members selected from a group consisting of an iron (Fe)-based catalyst, a cobalt (Co)-based catalyst, a nickel (Ni)-based catalyst, and a manganese (Mn)-based catalyst.

19. The method of claim 1, wherein the transition metal-based catalyst comprises an organometallic catalyst.

20. The method of claim 1, wherein the transition metal-based catalyst includes one or more members selected from a group consisting of a triphenylphosphine (TPP)-based ligand, a trialkylphosphine ligand, trialkylphosphite ligand, an organophosphate ligand, and a malononitrile ligand.

21. The method of claim 1, wherein the hydrogenation catalyst includes one or more members selected from a group consisting of platinum(IV) oxide (PtO₂), Pd supported on carbon (Pd/C), rhenium-nickel (Re—Ni) alloy, Raney nickel, Ni supported on silica-alumina (SiO₂—Al₂O₃), and nickel-aluminum (Ni—Al) alloy.
